(19) United States
(12) Patent Application Publication
(10) Pub. No.: US 2010/0098675 A1
(43) Pub. Date: Apr. 22, 2010

(54) METHODS FOR REDUCING THE SIDE EFFECTS OF OPHTHALMIC LASER SURGERY

(76) Inventor: NIKOLAI TANKOVICH, San Diego, CA (US)

Correspondence Address: STEMEDICA CELL TECHNOLOGIES, INC, 5375 MIRA SORRENTO PLACE, SUITE 100, SAN DIEGO, CA, 92121, US

(21) Appl. No.: 12/475,424

(22) Filed: May 29, 2009

Related U.S. Application Data
(60) Provisional application No. 61/038,711, filed on May 30, 2008.

(51) Int. Cl. A61K 35/14; A61K 35/12

(52) U.S. Cl. 424/93.73; 424/93.7

(57) ABSTRACT

A method for treating the side effects resulting from ophthalmic lasery surgery of the eye. The method administers stem cells as a means for treating the destruction of retinal and other tissues resulting from photocoagulation in the treatment of diabetic retinopathy and other eye disorders. Methods for using the invention with other ophthalmic laser surgical procedures, and other eye disorders, are also disclosed.

### PRIORITY

This application claims priority from provisional application Ser. No. 61/038,711 filed May 30, 2008, the entire contents of which are incorporated herein by reference.

### FIELD OF THE INVENTION

The invention relates to stem cell therapy. Specifically, the invention relates to the use of therapeutic stem cells in treating the damaging side effects of opthalmic laser surgery.

### BACKGROUND

Photocoagulation, also called laser treatment, is a group of therapeutic procedures that combine the use of a laser, opthalmic devices and lenses to create tiny burns on the retina. Photocoagulation may assume different methodologies depending on the condition that is being treated.

Focal macular laser therapy is one form of photocoagulation that is often used to treat disorders affecting the blood vessels of the macula, in particular diabetic retinopathy and retinal vein occlusion. The use of a laser in this application decreases the leakage from damaged blood vessels, helping to preserve normal retinal thickness and function and prevents further loss of vision.

Panretinal photocoagulation, or “scatter photocoagulation,” is used to treat abnormal retinal blood vessel growth (neovascularization) in diseases such as proliferative diabetic retinopathy or retinal vein occlusion. This procedure applies a laser in a “scatter” pattern to large areas of the peripheral retina which have poor blood flow (ischemia), and which release growth factors that exacerbate neovascularization. Untreated, retinal neovascularization often leads to bleeding in the eye (vitreous hemorrhage), traction retinal detachment, and/or neovascular glaucoma. After panretinal photocoagulation, the blood vessels tend to stabilize or regress. Since this treatment affects the function of the retinal periphery, some patients will recognize decreased peripheral and night vision. The size of the pupil and the central vision may also be affected in some patients.

Photocoagulation is also used to treat abnormal blood vessel membranes beneath the retina in age-related macular degeneration. Using photocoagulation to treat such conditions coagulates the blood vessel membranes and the vision in the area of treatment is permanently affected, and recurrences are common.

Photocoagulation may also be used to treat retinal tears or a small retinal detachments. In these applications, laser treatment is used to prevent further accumulation of fluid beneath the retina and minimize the risk of a vision-threatening retinal detachment. The laser is applied to the areas around the retinal defect. Over the course of a few weeks, the treated area develops a scar, which forms a tight seal between the retina and the underlying tissue. This procedure is sometimes performed around weak areas in the retina, such as lattice degeneration, in patients who may be at higher risk for retinal detachment.

Because therapeutic photocoagulation is used to burn part of the retina, many patients who receive this procedure experience damaging side effects including destruction of retinal tissue, ocular ischemia and vision impairment. Thus, there exists a need in the art for a method capable of treating the damaging side effects of laser therapy of the eye.

### SUMMARY OF THE INVENTION

The invention provides methods and compositions for using stem cell therapy to treat the harmful side effects of laser ophthalmic surgery.

One objective of the invention is to provide a method for treating a side effect resulting from the application of laser therapy in the treatment of an eye disorder comprising treating an eye disorder in a patient with a laser, wherein said treating produces a side effect in the eye of said patient, administering a therapeutic amount of stem cells to said patient.

A further objective of the invention is to provide a method for treating a side effect resulting from the application of laser therapy in the treatment of an eye disorder selected from the group consisting diabetic retinopathy, macular edema, age-related macular degeneration, retinal tears, retinal holes, retinal detachment, retinal vein occlusion, dot and blot hemorrhages, microaneurysms, exudates, glaucoma, vitreous hemorrhage, inflammatory optic neuropathies, post-cataract complications, endophthalmitis, infectious diseases, ocular ischemia syndrome, peripheral retinal degenerations, toxic retinopathies, tumors, choroidal tumors, choroidal disorders, vitreous disorders and traumatic injury.

A further objective of the invention is to provide a method for treating a side effect resulting from the application of laser therapy in the treatment of an eye disorder, wherein said side effect comprises vision impairment, destruction of retinal tissue, and/or bleeding.

A further objective of the invention is to provide a method for treating a side effect resulting from the application of laser therapy in the treatment of an eye disorder, wherein said said laser therapy comprises photocoagulation.

### BRIEF DESCRIPTION OF THE DRAWINGS

FIG. 1 shows a comparative evaluation of the degree of pigmentation of the area of laser damage from the rabbits of Example 1.

FIG. 2 shows a comparative estimate of proliferative vitreoretinal changes in kainate retinopathy in rabbits from Example 1 at the 30th day of observation.

FIG. 3 depicts changes in the amplitude of the ERG b-wave in kainate retinopathy and retrobulbar administration of NSPC.

FIG. 4 depicts changes in the amplitude of high-frequency RERG (32 Hz) of the ERG in kainate retinopathy and retrobulbar administration of NSPC.

FIG. 5 shows the results of a slit-lamp examination of eyes from Experiment 2, Day 1, after laser damage

FIG. 6 shows the results of a slit-lamp examination of eyes from Experiment 2, Day 4, after laser damage.

FIGS. 7-10 depict the slit-lamp examination of blood vessel coagulation of animals from Example 2, Group 2.

FIGS. 11-14 show the ERG dynamics after laser damage of the retina and treatment with the injection of olfactory ensheathing cells.

FIG. 15 shows the histological analysis of retinal tissue from an animal in Example 2.

FIGS. 16-17 are graphics showing the degeneration of retinal tissue in the animals from Example 2.

FIG. 18 is a retinogram of the eye of an animal from Example 3 three days after laser damage.

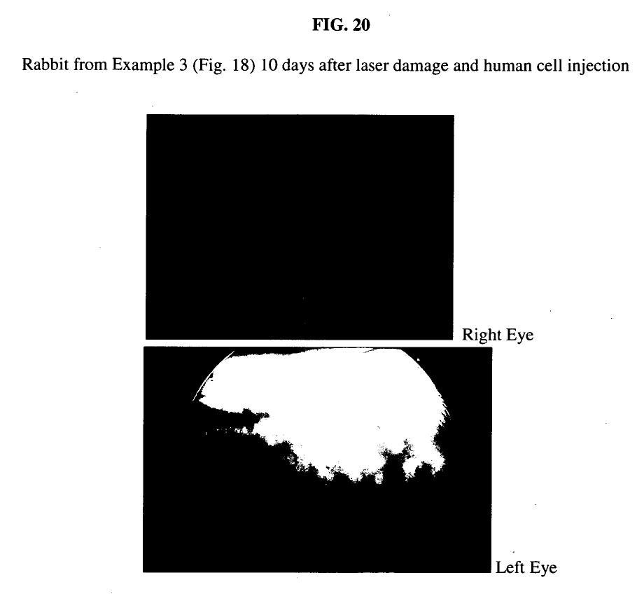

FIG. 19 is a retinogram of the eye of an animal from Example 3 seven days after treatment.

FIG. 20 is a retinogram of the eye of an animal from Example 3 ten days after treatment.

FIG. 21 is a retinogram showing the condition of the eye of an animal from Example 3 thirty days after treatment.

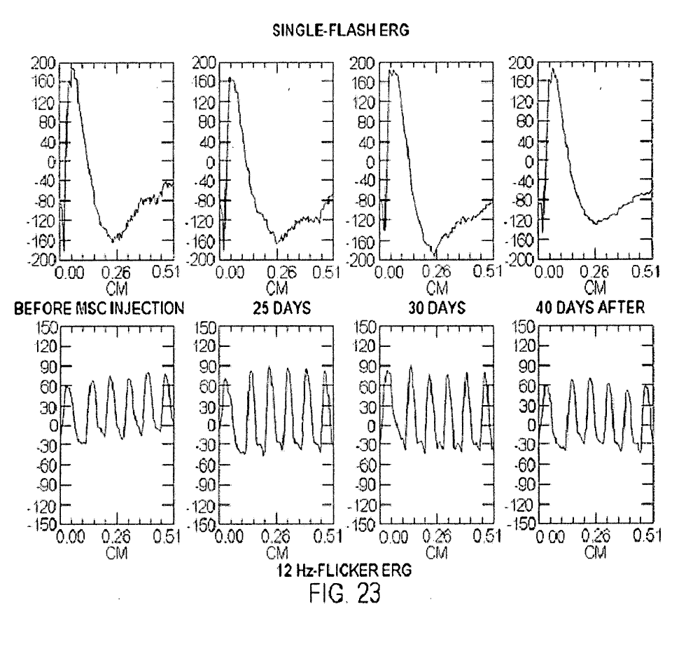

FIG. 22 is a retinograph depicting proliferative vitreoretinopathy.

FIG. 23 depicts the electroretinography results from single flash ERG and 12 Hz flicker ERG data recorded 25-40 days after laser damage and suprachoroid injection of human mesenchymal cells.

FIG. 24a-h are retinographs showing the results obtained from injecting human mesenchymal cells into the supra-choroidal space.

FIGS. 25a-f depict the electroretinography results obtained for Example 3.

FIGS. 26-27 depicts individual variations in ERG data obtained from the animals in Example 3.

FIGS. 28-29 depict the histological analysis of retinal tissue from an animal in Example 3.

FIG. 30 depicts Rabbit #27. A,B,C,D,E—small isolated laser burn spots. F—large deep lesion caused by coagulated artery (interrupted blood flow).

FIG. 31 shows Rabbit #28. Scale bar=200 micron. Typical small laser spot is shown. Arrows indicate spot that was included in analysis (the spot must not contain any photoreceptor cells).

FIG. 32 shows results from Rabbit #27. Same as Microphoto 30, but at higher magnification. Large secondary lesion caused by occluded small artery. Photoreceptor cells are gone. Scale bar=200 micron.

FIG. 32 shows Rabbit #27. A site of mesenchymal cell injection is shown by red arrow. Blue-stained mass is presumably formed by transplanted human cells.

FIG. 33 depicts a magnified portion of FIG. 32.

FIG. 34 depicts the results obtained for the intravitreal injection of mesenchymal stem cells with laser coagulation of the retina.

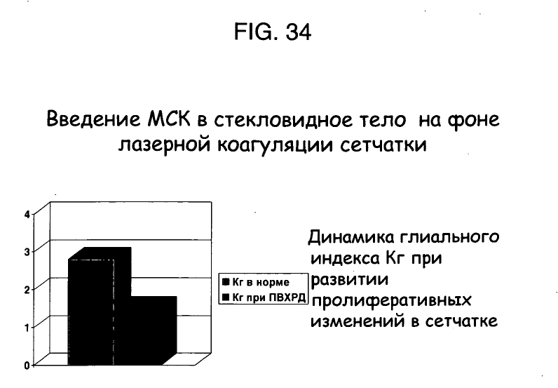

### DEFINITIONS

The term “opthalmic laser surgery” refers to any procedure wherein an eye disorder is treated by contacting one or more tissues of the eye with a laser. Photocoagualation therapy is a non-limiting example of ophthalmic laser surgery. “Opthalmic laser surgery” is used interchangeably with the terms “laser treatment of the eye,” “laser eye therapy,” “laser eye surgery,” “laser treatment” and “laser surgery.”

The term “eye disorder” refers to any disease, injury or condition that impairs the function of the eye. Some non-limiting examples of eye disorders include, but are not limited to, diabetic retinopathy (proliferative and non-proliferative diabetic retinopathy), age-related macular degeneration, retinal tears, retinal holes, retinal detachment, retinal vein occlusion, macular edema, neovascular glaucoma, vitreous hemorrhage and traumatic injury.

The term “photocoagulation,” refers to the condensation of protein material by the controlled use of an intense beam of light (e.g., laser). Photocoagulation may refer to the condensation of the retina by processes including, but not limited to, panretinal photocoagulation, focal photocoagulation and scatter photocoagulation.

The term “photocoagulation therapy” refers to the use of photocoagulation in the treatment of a bodily disorder including, but not limited to an eye disorder.

The term “side effect” refers to any adverse effect(s) resulting from opthalmic laser surgery. Side effects include, but are not limited to, loss or impairment of vision, ocular ischemia, and destruction of retinal tissue.

The term “ciliary body” is used to denote a tissue that resides between the peripheral regions of the retina and the iris, all of which arise from the same neuroepithelium during development.

The terms “reduce,” “ameliorate,” and “lessen” refer to the decrease, suppression, attenuatation, diminishing, arresting, or stabilizing of the development or progression of any eye disorder or ophthalmic laser surgery side effect.

The term “recruit” refers to the attraction of endogenous and/or exogenous stem cells into a tissue such as, for example, the retina or retinal pigment epithelium (RPE).

The term “regenerating the retina” as used herein refers to increasing the number, survival, function and/or proliferation of cells in the retina or retinal pigmented epithelium.

By “repairing retinal pigment epithelium damage” is meant ameliorating retinal pigment epithelium injury, damage, or cell death.

The term “stem cell” refers to an undifferentiated cell having the ability to both self-renew and differentiate to produce at least one functional, terminal cell type. A non-limiting example of a stem cell is a “precursor cell,” or “progenitor cell” which is a lineage-committed stem cell capable of dividing and differentiating to form a single, specific terminal cell type.

The term “mesenchymal cell” refers to a cell belonging to (i.e. originating from) the mesodermal germ lineage. Mesenchymal cells may be multipotent stem cells, precursor cells, or fully differentiated terminal cells having a specific functional phenotype.

“Multipotent,” or “multipotency,” refers to the ability of a stem cell to differentiate into various cells of one embryonic germ layer lineage (i.e the ectoderm, endoderm or mesoderm).

“Pluripotent,” or “pluripotency,” refers to the ability of a stem cell to differentiate into the various cells from each of the three embryonic germ layer lineages (i.e the ectoderm, endoderm and mesoderm).

“Regenerative” is used to refer to the ability of a substance to restore, supplement or otherwise rehabilitate the natural function of a tissue. This ability may be conferred by, for example, treating a dysfunctional tissue with regenerative cells. Regenerative cells treat dysfunctional tissue by replacing it with new cells capable of performing the tissue's natural function, or by helping to restore the natural activity of the dysfunctional tissue.

The term “pharmaceutically acceptable carrier,” or “carrier,” refers any of the well known liquid components useful for immunization such as, for example, culture media and phosphate-buffered saline. Some non-limiting examples of pharmaceutically acceptable carriers include, but are not limited to, those listed in Remington's Pharmaceutical Science (18.sup.th Ed., ed. Gennaro, Mack Publishing Co., Easton, Pa., 1990) and the Handbook of Pharmaceutical Excipients (4.sup.th ed., Ed. Rowe et al. Pharmaceutical Press, Washington, D.C.), each of which is incorporated by reference. “Pharmaceuticaly acceptable” means the carrier is non-toxic and does not cause an adverse reaction (e.g. an inflammatory or anergic reaction) when administered to a mammal.

The terms “treatment,” “treating,” “treat,” and the like, mean obtaining a desired pharmacologic and/or physiologic effect against a targeted disease, disorder, condition or injury. The term “treat” includes the reversing or otherwise improving a side effect resulting from, for example, ophthalmic laser surgery. “Treating” also refers to the therapeutic contacting of a laser with one or more tissues of the eye in a manner that reverses, slows the progression of, or stops an eye disorder.

The term “clone,” or “clonal cell,” refers to a single cell which is expanded to produce an isolated population of daughter cells (i.e. a “clonal cell population”).

The term “cell line” refers to one or more generations of cells which are derived from a clonal cell.

The term “derived from,” is used to indicate that a cell was obtained from a specific source such as, a tissue, a clonal cell line, a body fluid, a body structure, or a primary cell culture.

The term “isolated,” or “purifed,” refers to a homogenous, or essentially homogenous, population of donor cells that has been separated from its natural environment (i.e the donor). A population of cells is considered “purified,” or “substantially purified,” if it contains at least about 75% of a desired cell type. A clonal population of donor mesenchymal cells is a non-limiting example of a purified cell preparation.

“Retinal tissue” refers to the neural cells and associated vasculature that line the back of the eye. Structures within retinal tissue include the macula and fovea. Retinal tissue further includes the tissue that is juxtaposed to these neural cells (e.g. pigment epithelia) and associated vasculature.

The term “subject,” or “patient,” means a mammal, including, but not limited to, a human or nonhuman mammal, such as a bovine, equine, canine, ovine, or feline.

As used herein, the terms “prevent,” “preventing,” “prevention,” “prophylactic treatment” and the like refer to reducing the probability of developing a disorder or condition in a subject, who does not have, but is at risk of or susceptible to, developing the disorder or condition.

### DETAILED DESCRIPTION

The invention provides methods for using stem cell therapy to reduce the harmful side effects of ophthalmic laser surgery. In general terms, the invention is carried out by administering ophthalmic laser surgery in the treatment of an eye disorder in combination with the administration of a therapeutic amount of stem cells. The invention can therefore be interpreted as a staged process wherein laser therapy is used to treat an eye disorder, which is preceded, or followed by, the administration of a therapeutic amount of stem cells.

### Opthalmic Laser Surgery

The method of the invention may be used in connection with a variety of ophthalmic laser surgeries. This includes any procedure which produces a side effect which can be treated with the administration of stem cells as set forth in the present disclosure. Such ophthalmic laser surgery procedures include, but are not limited to, focal photocoagulation, scatter photocoagulation (panretinal photocoagulation), grid laser photocoagulation, endophotocoagulation, photodynamic therapy, argon laser trabeculoplasty (ALT), laser iridotomy, endoscopic cyclophotocoagulation (ECP), trabeculoplasty (e.g. selective laser trabeculoplassty) and combinations thereof.

The method of the invention may be used in connection with the laser treatment of a variety of eye disorders. These eye disorders include, but are not limited to, diabetic retinopathy (proliferative and non-proliferative diabetic retinopathy), macular edema, age-related macular degeneration, retinal tears, retinal holes, retinal detachment, retinal vein occlusion, dot and blot hemorrhages (tiny hemorrhages in the retina itself), microaneurysms (out-pouchings of capillaries), exudates (retinal deposits occurring as a result of leaky vessels), glaucoma (e.g. open-angle glaucoma and neovascular glaucoma), vitreous hemorrhage, inflammatory optic neuropathies, post-cataract complications, endophthalmitis, infectious diseases, ocular ischemia syndrome, pregnancy-related disorders, peripheral retinal degenerations, toxic retinopathies, tumors, choroidal tumors, choroidal disorders, vitreous disorders and traumatic injury.

In some embodiments, the invention is used to treat the side effects resulting from the treatment of proliferative diabetic retinopathy using photocoagulation. This includes the treatment of proliferative diabetic retinopathy using panretinal photocoagulation (PRP) and/or focal or grid laser photocoagulation. In general terms, these procedures involve contacting the back of the eye with a laser and causing “burns” which seal off damaged, leaking blood vessels that threaten vision. While these procedures produce a therapeutic effect by stopping the progression of neovascularization, they also have side effects including, but not limited to, destroying small areas of the retina which leads to impaired vision and the loss of the physical integrity of the retina. The administration of stem cells as set forth herein treats these side effects.

In some embodiments, the invention is used to treat the side effects resulting from the treatment of open-angle glaucoma using trabeculoplasty. With trabeculoplasty, a high-energy laser beam is used to shrink part of the trabecular meshwork, which causes other parts of the meshwork to stretch and open up allowing aqueous humor drain more easily from the eye.

In some embodiments, the method of the invention is used to treat side effects resulting from the use of photocoagulation in the treatment of retinal tears, retinal holes, and retinal detachment. Methods for using photocoagulation in the treatment of these conditions are known in the art, and include, those taught by U.S. Pat. No. 5,688,264, the disclosure of which is incorporated herein by reference. In general, these retinal conditions are treated by creating a controlled injury to the retinal pigment epithelium (RPE) and retina by means of photocoagulation to induce a chorioretinal adhesion (adhesion produced by wound healing) at the site of the retinal condition (e.g. tear, hole or detachment). Photocoagulation in the treatment of these conditions produces side effects including, but not limited to, destroyed retinal tissue and impaired vision. The method of the invention treats these side effects through the administration of stem cells as discussed below.

Although specific ophthalmic laser surgery procedures, and their side effects, are presently disclosed, one skilled in the art will appreciate that the invention may be used to treat any ophthalmic laser surgery side effect that is amendable by the administration of stem cells. Such side effects include, but are not limited to, the destruction of retinal tissue, blurred vision, impaired night vision, impaired peripheral vision, impaired color vision, post-lasering ischemia, blind spots and combinations thereof. Post-lasering ischemia refers to ischemia resulting from the destruction of the vasculature of the retina due to the therapeutic application of a laser.

### Stem Cells

The method of the invention administers stem cells as a means for treating the side effects of ophthalmic laser surgery. Accordingly, the method may be practiced with any stem cell, or combination of stem cells, that is capable of treating one or more side effects of ophthalmic laser surgery. The invention may be practiced with ectodermal cells, endodermal cells, mesenchymal cells, and combinations thereof.

Stem cells suitable for use with the invention may by pluripotent, multipotent or unipotent in terms of their plasticity. It is also contemplated that stem cells for use with the invention may be derived from syngeneic, allogeneic, or xenogeneic tissue sources. Xenogeneic tissue sources include, but are not limited to, non-mammalian vertebrates (e.g., avian, reptile and amphibian) and mammals. In preferred embodiments, the invention is practiced with syngeneic or allogeneic tissue sources. Suitable allogeneic tissue sources include tissues derived from living human donors and/or human cadavers.

In some aspects of the invention, the stem cells are derived from xenogeneic tissue sources including, but not limited to, primates, mice, pigs, bovines, cats, goats, rabbits, rats, guinea pigs, hamsters, horses, sheep, or a combination thereof. In a more preferred embodiment, the cells are primate cells, for example, cells from monkeys, orangutans, baboons, and combinations thereof.

In some aspects, the invention treats the side effects of ophthalmic laser surgery through the administration of mesenchymal cells. As used herein, the term “mesenchymal cell” refers to any cell that originates from the mesoderm germ layer. The potency of such mesenchymal cells may range from multipotent mesenchymal cells capable of forming different mesodermal cell types, down to fully differentiated terminal cells such as, for example, fibroblasts.

Mesenchymal cells for use with the invention may be derived from any source which provides mesenchymal cells capable of treating one or more ophthalmic laser surgery side effects. Mesenchymal cells may be derived from tissue sources including, but not limited to, bone marrow, adipose, peripheral blood, umbilical cord blood, umbilical cord, dermis, Wharton's jelly, hair follicle, periosteum, muscle tissue, uterine endometrium, amniotic fluid, tooth pulp, and combinations thereof. Mesenchymal cells may also be differentiated from pluripotent embryonic stem cells.

Mesenchymal cells for treating ophthalmic laser surgery side effects may comprise purified or non-purified cell populations. Purified mesenchymal cell compositions may be derived from clonal mesenchymal stem cells which have been expanded in culture. Alternatively, a purified cell composition may be derived from mesenchymal stem cells which have been isolated from a mixed population of cells, such as a fresh tissue preparation or a heterogeneous population of cells grown in culture. Methods for isolating mesenchymal cells from a mixed cell population are well known in the art and include, for example, FACS, cloning, density gradient centrifugation, magnetic sorting, affinity chromatography and serial passaging. Instructions for isolating mesenchymal stem cells suitable for practicing the invention are taught in the following references, the disclosures of which are incorporated by reference: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113; U.S. Pat. No. 6,387,367; U.S. Pat. No. 7,060,494; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

Mesenchymal cells for use with the invention may also comprise a non-purified (i.e. mixed) population cells. Such a mixed cell population may be obtained by, for example, combining two or more different purified mesenchymal cell types, or by culturing a mixed population of cells which has been expanded from a tissue sample (i.e. a primary cell culture). Suitable methods for preparing a primary culture of mesenchymal cells are known in the art, and include, for example, those disclosed in Zhang et al. Cell Biol Int. 2006 Nov. 29, Sakaguchi et al. Arthritis Rheum. 2005 August; 52(8):2521-9, Izadpana et al. J Cell Biochem. 2006 Dec. 1; 99(5):1285-97, Mareschi et al. J Cell Biochem. 2006 Mar. 1; 97(4):744-54, and Pozzi et al. Exp Hematol. 2006 July; 34(7):934-42, each of which is incorporated by reference. It is also contemplated that mesenchymal cells may be derived from an enriched population of cells obtained by the serial passage of a mixed population of cells (e.g. a primary cell culture). Still further contemplated are mixed populations of cells obtained by combining an already mixed cell population with either one or more purified cell types, or a second mixed population of cells.

Purified mesenchymal cells suitable for use with the invention are disclosed in the following publications, the disclosures of which are incorporated herein by reference: U.S. Pat. No. 5,654,186; U.S. Pat. No. 5,804,446; U.S. Pat. No. 5,980,887; U.S. Pat. No. 6,541,249; U.S. Pat. No. 6,676,937; U.S. Pat. No. 6,852,537; P.C.T. Pub. No. WO25112959; and U.S. Pat. Pub. No. 20060210544.

In some instances, it may be desirable to obtain a larger population of mesenchymal cells. Mesenchynmal cells may be serially expanded according to the teachings of the following references, the disclosures of which are incorporated by reference: Wu et al., Arch Dermatol Res. 2005 August; 297(2):60-7; Williams et al., Br J Dermatol. 1994 March; 130(3); Magerl et al. Exp Dermatol. 2002 August; 11(4):381-5; Inamatsu et al., J Invest Dermatol. 1998 Nov. 111(5):767-75; Warren et al., J Invest Dermatol. 1992 May; 98(5):693-9.

In some embodiments, the method of the invention treats ophthalmic laser surgery side effects through the administration of ectodermal cells. An ectodermal cell is any cell that belongs to the group of cells that develop from the ectoderm germ layer. Ectodermal cells for use with the invention may have varying degrees of plasticity. Examples of ectodermal cells include, but are not limited to; (a) multipotent cells capable of forming any cell type that originates from the ectoderm germ layer cell; (b) lineage-committed progenitor cells capable of dividing to form more progenitor cells or lineage-committed cells which differentiate into specialized, terminal cells; and (c) fully differentiated terminal cells that have a specific phenotype that supports a tissue function. Ectodermal cells for use with the invention include, but are not limited to retinal pigment epithelium (RPE) cells and precursors thereof, neural cells (e.g. glial, astroglial, oligodendrocytes, and neuronal cells) and precursors thereof, keratinocytes and precursors thereof. The invention also contemplates the administration of combinations of these cells.

In some embodiments, the method of the invention administers neural stem cells. Suitable neural stem cells may be derived from any source so long as the cells derived from that source provide a therapeutic effect when administered according to the methods disclosed herein. Neural stem cells for use with the invention may be derived from a variety of tissue compartments. In some embodiments, the neural stem cells are derived from nervous tissue. These neural-derived stem cells self-renew and remain undifferentiated in culture, assume a neural morphology upon introduction to mitogens in vitro, or upon introduction to the nervous tissue of an animal, and produce a regenerative effect when administered according to the methods disclosed herein. Neural tissue for providing a suitable source of neural stem cells includes (i) the peripheral nervous system, such as for example, the nasal epithelium, pigmented epithelium, non-pigmented epithelium, and ciliary body, (ii) the spinal cord, (iii) all the regions of the brain, including but not limited to, the forebrain, basal forebrain (cholenergic neurons), cerebellum, telencephalon, mesencephalon, hippocampus, olfactory bulb, cortex (e.g., motor or somatosensory cortex), striatum, ventral mesencephalon (cells of the substantia nigra), and the locus ceruleus (neuroadrenaline cells of the central nervous system), and (iv) combinations thereof. Instructions for deriving neural stem cells from nervous tissue are readily available in the art as shown by the following publications which are incorporated by reference: U.S. Pat. Nos. 5,750,376, 6,497,872, and 6,777,233; U.S. Pat. Nos. 5,196,315; 5,766,948, 5,968,829; 6,468,794, 6,638,763, 6,680,198, 6,767,738, 6,852,532, 6,897,061, 7,037,719; U.S. Patent Publication Nos. 20050112109, 20040048373, 20020039789, 20020039789, 20030095956, 20050118143, 20060148083, 20050074880, 20020086422, 20040253719, 20050003531, 20050125848, 20050142569, 20060099192 and 20060134280.

Neural stem cells for use with the invention may also be derived from non-neural (e.g. non-ectodermal) tissue sources. For example, stem cells capable of forming functional neural cells may be derived from mesenchymal stem cells. In some embodiments, this source of mesenchymal cells is the bone marrow. Such cells, in their undifferentiated state, assume a neural phenotype under in vitro conditions, or when introduced to the neural tissue of an animal. Amniotic fluid is another source of cells which can be differentiated into neural precursors. Instructions for deriving neural-potent bone marrow stem cells for use with the invention may be obtained from the following publication, which are incorporated by reference: U.S. Pat. Nos. 6,673,606 and 7,015,037; U.S. Patent Publication Nos. 20020164794, 20030003090, 20030039639, 20030059414, 20030203484, 20040151701, 20040208858, 20050282276, 20050249708, 20060105457, 20060177928; and Mareschi et al. Exp Hematol. 2006 November; 34(11):1563-72. In other embodiments, neural-potent mesenchymal cells are derived from umbilical cord blood. Suitable umbilical cord-derived cells, and their methods of isolation, are disclosed in U.S. Patent Publication Nos. 20020028510, 20050249708, 20040115804, 20050142118 and 20050074435, the disclosures of which are incorporated by reference. Neural-potent mesenchymal cells may also be derived from the scalp (i.e. skin) (see e.g. U.S. Patent Publication Nos. 20030003574, 20040253718 and 20040033597; and Shih et al. Stem Cells 2005 August; 23(7) 1012-1020), the peripheral blood (see e.g. U.S. Patent Publication Nos. 20040136973 and 20050221483), the placenta (see e.g. U.S. Patent Publication Nos. 20050089513 and 20060030039) and the amniotic layer (see e.g. U.S. Patent Publication No. 20030044977).

The neural stem cells for use with the invention may be made using purified or non-purified cells, as well as combinations of purified and non-purified cells. Non-purified compositions of neural stem cells may be obtained a number of ways. In some embodiments, the neural stem cell composition is made by combining separate, purified (i.e. isolated) neural stem cell populations. In other embodiments, the neural stem cell composition is obtained by culturing a mixed population of cells, such as a primary culture obtained from a tissue explant and expanded cell populations obtained therefrom. In still other embodiments, a non-purified composition of neural stem cells is obtained by combining one or more purified cell compositions, with a composition of mixed cell types such as a primary cell culture. Typically, primary cell cultures contain a mixture of cells as a variety of cells are able to grow in culture after being collected from an animal. Thus, primary cultures generally contain a combination of the different cell types which are able to proliferate in vivo. These cell types may have varying phenotypes (e.g. cellular markers) and varying levels of differentiation.

In some embodiments, the invention treats ophthalmic laser surgery side effects through the administration of fetal neural stem cells. Methods for producing primary cultures of fetal neural stem cells neural stem cells are widely available in the art. One such method is disclosed in U.S. Pat. No. 5,753,491, which describes the collection of fetal brain tissue from fetuses between 9-11 weeks of gestational age (7-9 weeks postconception). Following extraction, brain tissue is disassociated to produce a cell suspension which is subsequently plated on culture dishes and expanded under suitable conditions. Although the preparation of human fetal neural tissue is specifically called out here, one skilled in the art will appreciate that fetal neural stem cells may also be derived from both human and non-human post-natal nervous tissue. Fetal neural tissues suitable for deriving fetal neural stem cells include, but are not limited to, the telencephalon, diencephalon, forebrain, midbrain, cerebellum, pons and medulla, and spinal cord.

Examples of fetal neural stem cells suitable for use with the invention, and their methods of isolation include, but are not limited to: U.S. Pat. No. 6,852,532; U.S. Pat. App. 20020012903; U.S. Pat. App. No. 20020168767; Kallur et al. J Neurosci Res. 2006 Oct. 16 (Epub ahead of print); Eriksson et al. Exp Neurol. 2003 December, 184(2):615-35; Espinosa-Jeffrey J Neurosci Res. 2002 Sep. 15; 69(6):810-25; Kim et al. Exp Neurol. 2006 May; 199(1):222-35 Epub 2006 May 22; Englund et al. Exp Neurol. 2002 January; 173(1):1-21; Belicchi et al. J Neurosci Res. 2004 Aug. 15; 77(4):475-86; and Svendsen et al. Exp Neurol. 1997 November; 148(1):135-46. Fetal neural stem cells suitable for practicing the invention may be allogeneic or xenogeneic in nature. Neural stem cells for use with the invention need not be derived from a primary culture of mammalian neural tissue. Thus, it is also contemplated that the invention may be practiced with neural stem cells which are expanded from a clonal cell line. One cell line suitable for practicing the methods disclosed herein are taught in Flax et al. (Nat. Biotechnol. 1998 November; 16(11):1033-9), the disclosure of which is incorporated herein by reference.

Other methods suitable for producing a primary culture of neural cells are readily available in the art. The following publications, which are incorporated by reference, provide the teachings necessary to enable one skilled in the art to prepare a primary culture of neural stem cells for use with the invention: U.S. Pat. Nos. 5,750,376, 6,497,872, and 6,777,233; U.S. Patent Publication Nos. 20050112109, 20040048373, 20020039789, 20020039789, 20030095956, 20050118143, 20060148083, and 20050074880; Isolation, Characterization and Use of Stem Cells from the CNS, 18 Ann. Rev. Neurosci. 159-92 (1995); M. Marvin & R. McKay, Multipotential Stem Cells in the Vertebrate CNS, 3 Semin. Cell. Biol. 401-11 (1992); R. P. Skoff, The Lineages of Neuroglial Cells, 2 The Neuroscientist 335-44 (1996). A. A. Davis & S. Temple, A Self-Renewing Multipotential Stem Cell in Embryonic Rat Cerebral Cortex, 362 Nature 363-72 (1994); A. G. Gritti et al., Multipotential Stem Cells from the Adult Mouse Brain Proliferate and Self-Renew in Response to Basic Fibroblast Growth Factor, 16 J. Neurosci. 1091-1100 (1996); B. A. Reynolds et al., A Multipotent EGF-Responsive Striatal Embryonic Progenitor Cell Produces Neurons and Astrocytes, 12 J. Neurosci. 4565-74 (1992); B. A. Reynolds & S. Weiss, Clonal and Population Analyses Demonstrate that an EGF-Responsive Mammalian Embryonic CNS Precursor is a Stem Cell, 175 Developmental Biol. 1-13 (1996); Cattaneo et al., Mol. Brain. Res., 42, pp. 161-66 (1996); and B. P. Williams et al., The Generation of Neurons and Oligodendrocytes from a Common Precursor Cell, 7 Neuron 685-93 (1991).

Although fetal neural stem cell compositions are called out above, the inventive method may also be practiced with cells derived from adult neural tissue (e.g. tissue explant or cadaver source). Adult neural stem cells suitable for treating ophthalmic laser surgery side effects, and methods of deriving them, are taught in the following publications, the disclosures of which are incorporated by reference: U.S. Pat. Nos. 5,356,807, 5,851,832, 6,638,763 and 6,812,027; and U.S. Patent Publication Nos. 20030049234, 20030095956, 20030118566, 20040253719, 20050112109 and 20050118143.

Neural stem cells for use with the invention may be derived from human heterologous sources including fetal tissue following elective abortion, or from a post-natal, juvenile or adult organ donor. Autologous neural tissue can be obtained by biopsy, or from patients undergoing neurosurgery in which neural tissue is removed, for example, during epilepsy surgery, temporal lobectomies and hippocampalectomies. Neural stem cells have been isolated from a variety of adult CNS ventricular regions, including the frontal lobe, conus medullaris, thoracic spinal cord, brain stem, and hypothalamus, and proliferated in vitro using the methods detailed herein. In each of these cases, the neural stem cell exhibits self-maintenance and generates a large number of progeny which include neurons, astrocytes and oligodendrocytes.

In addition to the use of primary cultures of neural stem cells, the method of the invention contemplates of the use of purified neural stem cells. Purified neural stem cells for use with the invention may be derived using methods well known in the art, such as for example, FACS, magnetic sorting, serial passaging, cloning, and affinity chromatography. Such neural stem cells may be purified from a tissue explant or a mixed population of cells grown in culture. Suitable purified cell populations, and their methods of preparation, are disclosed in the following publications, the disclosures of which are incorporated by reference: U.S. Pat. Nos. 5,196,315, 5,766,948, 5,968,829, 6,468,794, 6,638,763, 6,680,198, 6,767,738, 6,852,532, 6,897,061 and 7,037,719; and U.S. Patent Publication Nos. 20020086422, 20040253719, 20050003531, 20050125848, 20050142569 and 20060099192.

In some embodiments, the method of the invention treats the side effects of ophthalmic laser surgery by administering olfactory ensheathing cells. Olfactory ensheathing cells (OEC) are a specialized type of glia that guide primary olfactory axons from the neuroepithelium in the nasal cavity to the brain. OEC are unique glia found only in the olfactory system that retain exceptional plasticity, and support olfactory neurogenesis and the re-targeting across the PNS:CNS boundary in the olfactory system. The primary purpose of the olfactory ensheathing cells is to induce remyelination of damaged nerves, as well as to allow for regeneration in specific circumstances. Methods for deriving OEC for use with the invention are taught in the following references, the disclosures of which are incorporated herein by reference: C R Biol. 2007 June-July; 330(6-7):557-60. Epub 2007 May 9; Brain Res Rev. 2007 November; 56(1):236-58. Epub 2007 Aug. 14; J. Neurotrauma. 2007 November; 24(11):1773-92; and Glia. 2007 February; 55(3):303-11.

Cells for use with the invention, as disclosed herein, may be genetically modified to express a gene of interest (i.e., a gene that encodes a molecule having a desired biological activity). Such genes can be inserted into a cloning site of a suitable expression vector by using standard techniques. The expression vector containing the gene of interest may then be used to transfect the desired cell line. Standard transfection techniques such as calcium phosphate co-precipitation, DEAE-dextran transfection or electroporation may be utilized. Commercially available mammalian transfection kits may be purchased from e.g., Stratagene. Transgenic-mouse-derived cell lines can also be used. See, e.g., Hammang et al., Methods in Neurosci., 21, p. 281 (1994).

It is also contemplated that the cells for use with the invention as disclosed herein may be conditionally-immortalized cells. Such cells include cells engineered with chimeric genes composed of an oncogene under the direction of an inducible promoter element.

### Administration of Stem Cells

Stem cells may be administered using any administration route that allows the stem cells to treat an ophthalmic laser surgery side effect. In some aspects of the invention, stem cells are injected directly into the eye of a patient. Suitable injection routes include, but are not limited to, intraocular routes such as retrobulbarly, subconjuctivally, intravitreally, suprachoroidally, and subretinally. These routes may be used singularly, or in combination with one another. It is also contemplated that stem cells may be administered systemically (e.g. intravenous injection), topically, and/or periocularly. These routes may be used alone or in combination with the intraocular routes of administration discussed above.

One aspect of the invention relates to the preparation of stem cells for administration. In some embodiments, the stem cells are administered using a pharmaceutically acceptable carrier (i.e. “carrier,” or “pharmaceutical carrier”). Suitable carriers for use with the invention include, but are not limited to, those disclosed in the following references, the disclosure of which are incorporated herein by reference: 18.sup.th Ed., ed. Gennaro, Mack Publishing Co., Easton, Pa., 1990; and the Handbook of Pharmaceutical Excipients (4.sup.th ed., Ed. Rowe et al. Pharmaceutical Press, Washington, D.C.).

One skilled in the art will understand that the selection of a pharmaceutical carrier will depend upon the particular administration route to be used.

Another aspect of the invention relates to the timing of the administration of stem cells. Stem cells may be administered before the administration of ophthalmic laser surgery. Thus, for example, stem cells may administered up to ______ days before a patient undergoes ophthalmic laser surgery. It is also contemplated that stem cells may be administered after a patient undergoes ophthalmic laser surgery. Stem cells may be administered, for example, up to ______ days after a patient undergoes ophthalmic laser surgery. In a preferred embodiment, stem cells are administered ______ after laser surgery. Stem cells may be administered in a single dose, or in a series of administrations.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

### Example 1

### Administration of Neural Stem Cells in the Treatment of Laser and Chemical Induced Retinal Damage

### Materials and Methods

### Tissue Isolation

Used as donor material were tissues of the forebrain of 7-12-week-old human embryos obtained from clinically healthy women artificially terminating pregnancy at periods corresponding to WHO recommendations accepted by the Ministry of the Russian Federation (Article No. 36, Order of the Ministry of Health No. 302 from Dec. 28, 1993). Aborted material was placed in a thermal container (4° C.) and delivered to a tissue culture laboratory no later than 5 hours after artificial termination of pregnancy. Fetuses free of visible developmental defects were treated with a disinfectant solution and transferred to cool F-12 medium (4° C.), after which the brain was totally isolated. The isolated tissue fragments were placed in petri dishes (60 mm, Costar, United States) containing 2 mL of F-12 medium and mechanically dissociated by multiple pipeting until a homogeneous cell suspension was obtained. Undissociated tissue fragments were removed by sedimentation. A cell count was performed in a hemocytometer, and cell viability was determined by Trypan Blue and propidium iodide exclusion test in a flow cytofluorometer. Suspensions with a viability of at least 60% were used for culturing.

### Cell Culturing

Two media were used for culturing: neural progenitor basal medium (Clonetics, United States) with the addition of the standard set of growth factors, including fibroblast growth factor (bFGF), neural survival factor (NSF), epidermal growth factor (EGF), and gentamicin/amphotericin, and a medium based on Dulbecco's minimal medium (DMEM) and F-12 medium (in a 1:1 ratio), complement N2 (1:100), FGF (20 ng/mL), EGF (20 ng/mL), and heparin (8 μg/mL). A suspension of cells (2 million/mL) was cultured in the above media for 12-60 days at 37° C. in an atmosphere with 5% CO₂. In both media, by the second day the formation of freely floating cell aggregates was observed, which increased in size during culturing and eventually formed the spheroid structures neurospheres containing several tens of cells with a viability of 98-100%.

### Immunocytochemical Analysis

After 14 days of culturing, a portion of the freely floating cell aggregates and/or neurospheres were fixed for histological, immunocytochemical, and electron microscopic studies. Cultured cells were fixed at +4° C. over 5-7 hours with 4% paraform prepared in phosphate buffer. After fixation, cells were washed, processed with 3% Triton-X, and washed again, after which they were incubated for 12 hours with primary antibodies. The following antibodies were used: human anti-nestin (Chemicon, 1:30), anti-glial fibrillary acidic protein (GFAP) (DAKO, 1:250), (Abcam, 1:200), anti-vimentin (NeoMarkers, 1:100), and anti-neurofilament-70 (Serotec, 1:500). Then, cells were processed with a solution of secondary antibodies (Vector Laboratories, 1:200) for 1 hour. In cases of double staining, cells were processed with secondary antibodies labeled with Cy2 and Texas Red fluorescent dyes, anti-rabbit and anti-mouse, respectively (Jackson). Such staining enabled the visualization of two antibodies in a single preparation, for example, GFAP and β-III-tubulin or nestin and vimentin.

### Characterization of the Culture

Light microscopy of semithin sections of neurospheres stained with toluidine blue, as well as their electron microscopic study, revealed cells at different stages of mitotic division.

Immunocytochemical studies showed that, over the period of culturing, neurospheres acquired a heterogeneous cell composition, as some of SC began spontaneous differentiation along the neural and glial pathways. Nestin-immunopositive cells had small rounded or elongated bodies with a small number of processes. A thin border of nestin-immunopositive cytoplasm was seen around the unstained nucleus and processes of these cells. GFAP-immunopositive cells had more intensely stained bodies, which apparently was due to a thicker layer of cytoplasm. Cells containing (β-III-tubulin had intensely stained bodies and long processes. GFAP- and β-III-tubulin-immunopositive cells were located in inner areas of neurospheres. As a rule, these cells formed compact groups.

Before transplantation, the cell suspension was thoroughly washed free of the culture medium, the number of viable cells was counted by the trypan blue exclusion test, and cells were suspended in a minimal quantity of physiological solution. An NSPC suspension can be stored in this form at 4-25° C. for 2-3 hours before transplantation.

### Cryopreservation of NSPC

For wide use of transplantation of NSPC in opthalmological practice, the availability of sufficient reserves of cells prepared in advance and having a high level of preservation is of principal importance. This problem can be solved using the method of low-temperature preservation, which allows biomaterial to be preserved suitable for transplantation for many years.

In this work, studies were performed with two models of experimental damage to the retina: laser-induced and toxic (kainate-induced).

### Laser Damage

Laser damage to the rabbit retina was carried out with an argon laser. Coagulation was performed in the lower half of the ocular fundus through a face of a three-mirror lens (20 spots per experimental eye). The average radiation power was 400 mW, the duration of exposure was 0.1 s, and the size of the spot was 100 μm. According to the L'Esperance classification, the radiation burn under such conditions corresponds to the third degree. Two series of experiments were performed with this model. In the first series (20 rabbits—40 eyes), we studied the effect of retrobulbar NSPC administration on retinal reparation. After laser trauma, NSPC in a dose of 8 million cells in 1 mL of physiological solution were administered into the retrobulbar space of the right eyes; in the same way, 1 mL of physiological solution was injected into the left (control) eyes. The choice of the method was dictated by its simplicity.

In the second series (20 rabbits—40 eyes), we studied the capability of NSPC to survive, migrate, and integrate into the retina of the recipient upon transplantation into the suprachoroidal space (SCS). After laser damage to the retina was carried out, NSPC were transplanted into both eyes into the SCS in the form of a suspension in a dose of 500,000 to 1 million cells in 0.05 mL of physiological solution using a Hamilton microsyringe. For further visualization of the administered cells in histological preparations, cells were stained with the fluorescent nuclear dye bisbenzimide (Hoechst 33342, Sigma) before transplantation.

### Toxic Retinopathy

Toxic retinopathy was modeled by intravitreal administration of kainic acid (Sigma). Four series of experiments were performed with this model.

In series I (6 rabbits, 12 eyes), the dose of the administered toxin was worked out.

In series II (15 rabbits, 30 eyes), after the administration of kainic acid into the vitreous body of both eyes, NSPC were intravitreally injected into the right eyes (the experimental group) at a dose of 3 million cells in 0.1 mL of physiological solution. A dose of 0.1 mL of physiological solution was administered in the same way into the left eyes (the control group).

In series III (4 rabbits—8 eyes), NSPC were transplanted intravitreally at the same dose into the right eyes (the experimental group) 5 days after modeling of kainate retinopathy. Physiological solution in a volume of 0.1 mL was administered into the left eyes. In the experiments, marked inhibition of electrophysiological indices of the retina was observed on second day after cainate treatment. The late transplantation pursues the aim of studying the effect of NSPC on reparative processes and the functional activity of the retina with pronounced electrophysiological changes, which is clinically justified.

In series IV (10 rabbits, 20 eyes), we studied the effect of retrobulbar administration of NSPC. After modeling of retinopathy in the right eyes (the experimental group), NSPC were injected into the retrobulbar space in a suspension containing 10 million cells in 1 mL of physiological solution. A dose of 1 mL of physiological solution was administered in the same way into the left eyes (the control group). Injections were repeated once a week for a total of four injections over 1 month.

The results of experimental series II, III, and IV allowed us to compare the clinical and functional changes in the retina in the model of kainate retinopathy for the different methods of NSPC transplantation (intravitreal and retrobulbar), as well as to evaluate the effectiveness of NSPC transplantation in the early and late periods of experimental retinopathy.

### Clinical Observation

Clinical observation of the state of the eyes was performed using focal and side illumination for general examination of the anterior section of the eyes, biomicroscopy with a slit lamp (Carl Zeiss Jena), and opthalmoscopy with a direct opthalmoscope (HEINE).

Clinical evaluation of the state of the ocular fundus for the model of laser damage to the retina was performed according to a point system developed characterizing the degree of pigmentation of coagulates:

TABLE 1

| Score | Degree of pigmentation in the region of laser damage |
|---|---|
| 1 | Up to ¼ of the area of damage |
| 2 | ¼-¾ of the area of damage |
| 3 | More than ¾ of the area of damage |

Clinical evaluation of the state of the ocular fundus in the experimental series for the model of kainate retinopathy, namely, the extent of proliferative changes, was performed according to a point system developed:

TABLE 2

| Score | Intensity of clinical symptoms |
|---|---|
| 0 | Absence of visible changes |
| 1 | Slight cloud-like preretinal turbidity |
| 2 | Initial fibrotic changes of the type of vitreoretinal lattice |
| | degeneration |
| 3 | Pronounced fibrotic changes with growth of gliotic tissue |

### Electroretinographic Studies

Electroretinographic studies were carried out on a Retinograf device (Infobiotek, Russia). Biopotentials were recorded using a contact lens with an electrode (a silver wire with a cotton wick soaked in physiological solution) mounted in it. Thin subcutaneous needles served as the reference and grounding electrodes. Using a spherical Ganzfeld stimulator, the Ganzfeld ERG (0.25 Hz, 1 μJ/cm²) and rhythmic ERG (RERG) were recorded at a flicker rate of 12 and 32 Hz (0.36 μJ/cm²) and the glial index (C_{G}) was calculated as the ratio of the amplitude of the b-wave of the ERG and the low-frequency RERG. The studies were carried out before modeling of retinopathy and administration of SC and 1, 3, 7, 20, and 30 days after the injections.

### Morphological and Immunohistochemical Analysis

With the model of laser damage to the retina, a morphological and immunohistochemical study was carried out on enucleated eyes of rabbits 1, 5, 10, 20, and 30 days after trauma and NSPC transplantation. Eyes were fixed with 10% formalin in phosphate buffer and then subjected to standard histological processing. Semithin sections (1 μm) were stained with hematoxylin-eosin.

For the immunohistochemical study, material was fixed in 4% paraform in phosphate buffer (pH 7.3) and then washed with 30% sucrose at 4° C. Sections 20 μm thick were made in a cryostat and then mounted on slides. Sections with transplanted cells were selected based on the fluorescence of cells stained with bisbenzimide. Sections were stained with primary antibodies to human cell nuclei (Chemicon, 1:30) and human cell nestin (Chemicon, 1:20) and then processed with a solution of secondary antibodies (Vector Laboratories) diluted to 1:200 for 1 hour. After being washed three times, sections were clarified with a 50% solution of glycerol in phosphate buffer and examined in an Opton fluorescence microscope. Histological material was photographed with a Nikon 4500 digital camera.

### Results and Discussion

The results of series I of the experiment with the model of laser damage to the retina showed that, in the clinical study of the anterior and posterior sections of the eye, no signs of an inflammatory or allergic reaction were revealed. This is due to the fact that transplanted cells, like any embryonic and fetal tissues, cause no immune conflict because they are formed in the immature immune system and have weakly expressed histocompatibility antigen complexes (MHC I and MHC II) (Vasil'ev, N. V. et al., 1996); this decreases the level of posttransplant complications by an order.

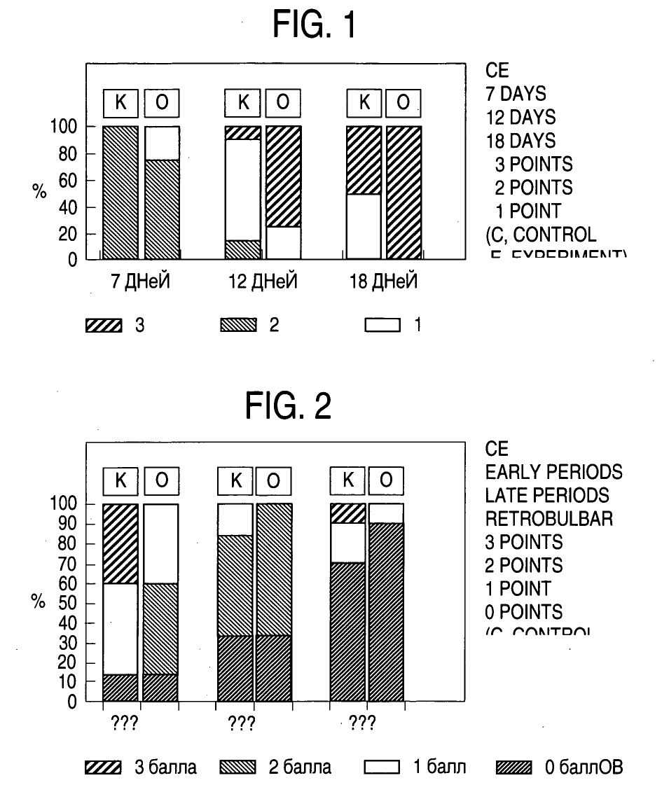

Opthalmoscopy revealed that in the experimental group retinal edema in the zone of laser damage disappeared on average after 3 days, and in the control group, after 4 days; the appearance of pigment in coagulated areas began in experimental eyes from the fourth to fifth day, and in control eyes, from the sixth to seventh day. Pigmentation of areas of laser damage treated with NSPC was completed on average after 16-17 days, and on administration of physiological solution, on average after 19-20 days. Thus, in the experimental group, a faster pigmentation of coagulates, on average by 2-3 days, was noted. On the 18th day of observation, full pigmentation of the area of laser burn was present in all experimental eyes, and in the control group, only in 50% of animals (see FIG. 1).

### Histological Examination

Histological examination found a significant difference between control and experimental eyes: migration of pigment cells into the inner layers of the retina was noted in control specimens where aggregates formed and chorioretinal adhesion was rougher. In the experimental eyes, the pigment epithelium proliferated into several layers within the limits of retinal external layers, and more complete reconstruction of damaged areas was noted. The obtained results of clinical and morphological observations indicate an intensification of reparative regeneration of the retina upon transplantation of NSPC.

Such a stimulating effect is due to the fact that SC release various biologically active substances: neurotrophic factors, growth factors, key metabolites, stage-specific proteins, and antioxidants that activate the regeneration of damaged tissues of the recipient (Repin, V. S. 1998; Sukhikh, G. T. and Malaitsev, V. V., 2001).

### Immunohistochemical Examination

Immunohistochemical study conducted in series II of the experiment on days 1, 5, 10, and 30 found transplants of cultured human NSPC in the eyes of all experimental animals. Positive staining of preparations with fluorescent emission of cells stained with bisbenzimide and anti-human nuclear antibodies specifically identified the administered human cells. Antibodies to human nuclei always clearly revealed the administered NSPC and served as a good marker of human cells in the rabbit eye.

The distribution density of transplanted cells decreased with distance from the site of administration. Cells migrated towards the posterior pole of the eye, reaching the zone of laser trauma on the 10th day after transplantation. Transplanted cells, immunopositive for human cell nuclei, were distributed in groups or singly. Reaching the zone of laser damage, cells of transplants were located in the choroid and in external and internal layers of the retina.

Double immunohistochemical staining using antibodies to human cell nuclei and human nestin showed that, among transplanted cells, it is truly SC that are preserved. Individual nestin-positive cells migrated from the site of administration along the tissues of the choroid and retina.

These data suggest that the transplanted cells can survive for 30 days and migrate to a zone of pathological changes, in several cases, preserving their multipotent status. In opthalmology, stem cells are administered, as a rule, subretinally or intravitreally. It is known that the choroid is loosely bound to the scleral brown lamina by a system of long and short connective tissue laminae, which are easily destroyed mechanically. This peculiarity of the anatomy of SCS allows various biomaterials to be used for surgical treatment of senile macular degeneration. It seems that the administration of cells to the SCS of the eye is less traumatic for the retina and, moreover, ensures freer migration of transplanted cells. The movement of cells to the posterior pole of the eye toward the area of laser trauma confirms the necessity of damage, which is a “signal” for of SC activation. Thus, the transplantation of cells to the SCS of the eye is also possible in the clinic.

In study of the effects of transplantation of stem cells into the eyeball, the determination of the nature of their influence on the structure and function of the retina, as well as an objective evaluation of the functional ability of the regenerated neurons, is of great significance. However, in the numerous works published on the problem of stem cells, electrophysiological studies are completely absent because of the difficulties in performing them in rodents. In isolated reports, visual functions were studied in psychophysical behavioral experiments without the use of objective criteria for evaluating the activity of sensory tissue (Lund, R. D. et al., 2001).

### Induced Kainate Retinopathy

For possible study of the functional state of the retina in transplantation of SC, for the first time in rabbits a model of toxic damage to the retinal membrane—kainate retinopathy—was created. Kainic acid is an excitatory amino acid and a direct agonist of the glutamate receptor. The administration of this toxin in situ leads to excess accumulation of extrcellular glutamate, which in large doses has a neurotoxic effect, blocking synaptic transmission between photoreceptors and biopolar cells and causing the death of the latter (Chidlow, G. and Osborn, N. N., 2003). The use of this model of retinopathy is justified from the point of view of the possibility of studying the effect of NSPC on electrogenesis of the retina and, consequently, on the visual functions.

In series I of the experiment with the model of kainate retinopathy, the appropriate dose of the toxin was found. A dose of kainate of 0.04 mg, which turned out to be optimal both for electrophysiological studies and for analysis of funduscopic changes, was used in further studies. Clinical evaluation of the state of the ocular fundus was carried out according to a point scale we developed that reflected the intensity of proliferative vitreoretinal changes. In the series with the administration of NSPC in the early periods, in the control group pronounced fibrotic changes in the retina of the type of vitreoretinal lattice degeneration were observed by the 20th day—2 points on the proposed scale in 86.7% of eyes; in the experimental group, these changes were observed only in 40% of cases. By the 30th day, a significant difference was revealed between the experimental and control groups; in the eyes containing the cell transplants, the picture remained unchanged; in the control, symptoms of toxic damage to the retina had progressed: in 40% of eyes, pronounced fibrotic changes in the retina with the growth of glial tissue developed (3 points).

In the series with late transplantation of NSPC, in the control group proliferative changes progressed to 2 points on day 20 in 16.7% of cases; in the experimental group, such changes were not observed: in 50% of cases, changes were absent, and in 50% they remained on the level of slight preretinal turbidity (1 point).

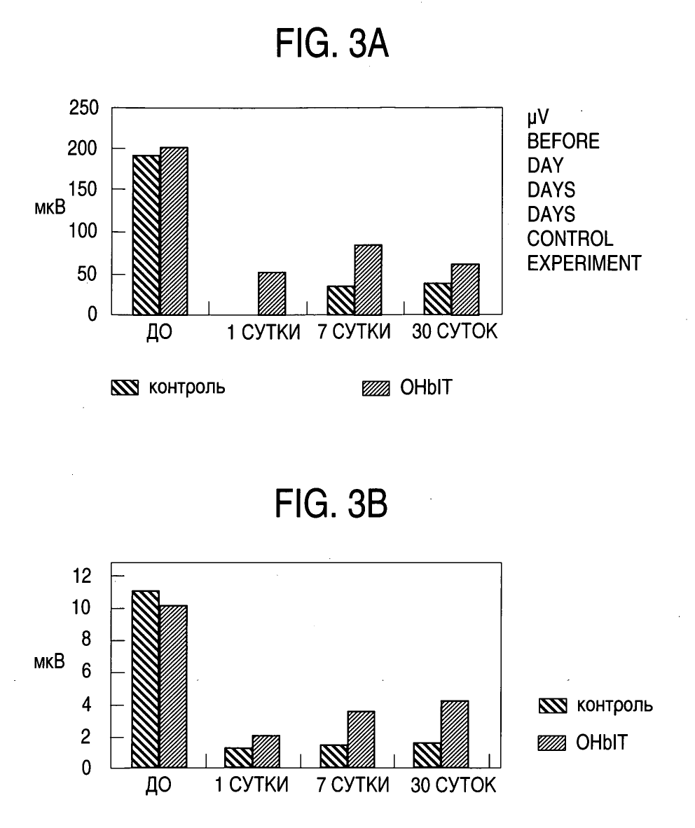

In series IV with retrobulbar injection of NSPC, we found regression of initial fibrotic changes in a number of cases both in the control and experimental group. Nevertheless, at the 20th day in the experiment, proliferative changes had reached an estimate of 2 points in only one animal, while in the control there were two such cases; in one of these, the development of gliosis was observed by the 30th day (3 points) (see FIG. 2). Pathological changes developed, as a rule, in the central parts of the ocular fundus, under the optic disc, and involved ⅛-⅕ of the area of the ocular fundus.

Thus, analysis of the clinical observation data on the state of the ocular fundus in all three variants of NSPC transplantation (intravitreal administration in early and late periods after modeling of retinopathy and retrobulbar administration) revealed a lower degree of proliferative vitreoretinal changes in experimental eyes. The development of glial tissue (3 points) 1 month after the beginning of the experiment was not observed in even one eye with transplanted cells in any NSPC administration variant, while in the control group, pronounced fibrotic changes developed with growth of glial tissue in 7 cases (40%) upon intravitreal administration in early periods and in 1 case (10%) upon retrobulbar administration (FIG. 2).

The most significant difference between control and experimental eyes was found in the group with intravitreal administration of NSPC in early periods—within 30 min after the injection of kainic acid. The goal of this experimental series was to reveal the ability of to prevent the development of a pathological process in the retina. The obtained results showed a positive neuroprotective effect of transplanted cells, probably due to the release of biologically active substances.

In the group injected with NSPC 5 days after the modeling of kainate retinopathy (late periods), transplantation of NSPC was less effective. This can be explained by the fact that a significant number of second- and third-order neurons die within 5 days.

According to literature data, the complete destruction of neurons occurs within 48 hours after administration of kainic acid to the striatum of the rat brain, (Coyle, J. T. et al., 1978; Simon, G. R. et al., 1976). Nevertheless, in this work, we found intense proliferation (2 points) in 1 case out of 6 in control samples, whereas changes found in experimental samples were confined to the initial stage (0-1 point) in all cases.

### Electroretinographic Studies

Electroretinographic studies of series I of the experiment with kainate retinopathy found degenerative changes in second-order neurons (ON bipolar cells) with a preserved function of first-order neurons (photoreceptors), which is supported by literature data (Chidlow, G. and Osborn, N. N., 2003). Low-frequency RERG objectively reflects the overall function of neurons of the inner nuclear layer, primarily ON bipolars. The gross reduction in its amplitude with a preserved ERG a-wave, reflecting the function of photoreceptors, indicates the degeneration of bipolar cells.

The practically complete disappearance of the ERG b-wave, in whose generation not only bipolar but also glial cells participate, suggested that kainate exerts a very great and, possibly, primary effect on the neuroglia. According to data of fundamental pathophysiology (Riechenbach, A., 1998; Bringmann, A., 2000; Zueva, M. V., 2002), in the normal mature retina, Müller cells have a high membrane potential and have a potential-dependent ability to regulate the level of glutamine synthetase and to remove an excess of glutamate from the extracellular space. Müller cells synthesize and secrete glutamine synthetase and have developed mechanisms for utilizing glutamate from the extracellular medium and for its inactivation.

Damage to retinal tissue decreases the membrane potential of Müller cells and reduces their ability to ensure potential-dependent absorption of glutamate from the extracellular space, its inactivation and recycling and, consequently, the generation of the ERG b-wave. The disappearance of the b-wave in kainate-induced retinopathy reflects the marked suppression of Müller cells, which, apparently, is the reason for the disturbance in their potential-dependent ability to regulate the level of glutamine synthetase and recycle glutamate. Thus, the results suggest the significant involvement in kainate retinopathy of the Müller glia as well.

Electroretinographic studies in further experiments showed that the most complete functional rehabilitation of the retina took place in the experimental group with the early administration of NSPC, which is completely natural, taking into account the transplantation of donor material at the early stages of development of retinopathy.

We found that the virtually simultaneous administration of neurotoxin and NSPC prevents the manifestation of kainate retinopathy, protecting the retinal cell types that undergo the greatest change upon injection of kainic acid. In the control group, in 53.3% of rabbits during the entire period of observation, the b-wave was either completely absent or sharply reduced, and a pure RIII component was recorded or a “minus” negative ERG: a b-wave below the isoelectric line with a preserved a-wave. In the remaining 46.7%, a “plus” negative ERG was generated: the b-wave is reduced, but is above the isoelectric line and has a longer latency.

In the experimental group after 1 day, a low-amplitude (55.0±14.7 μV (M±m)) b-wave was observed. Subsequently its amplitude progressively increased and by the 30th day of observations it amounted to 128.0±18.0 μV on average (initial values amounted to 135.0±12.9).

The results of this study demonstrate the preservation or restoration under the influence of stem cells of some functions inherent to mature Müller glial cells, primarily the absorption, inactivation, and recycling of glutamate and the generation of the ERG b-wave.

The functional activity of photoreceptors, estimated by the amplitude of the ERG a-wave, in both eyes remained practically the same. In connection with this, the differences obtained in the dynamics of the low-frequency RERG in the control and experimental eyes can be completely explained by differences in the functional reaction of bipolar cells. The increase in the amplitude of the RERG in the experimental group to values of 28.0±9.9 μV by the 30th day of observations in comparison with the control group (6.5±3.6 μV) can reflect a significantly better preservation of second-order neurons.

It is known that Müller cells—highly specialized glial elements—in addition to their supporting and insulating functions, actively transport metabolites at different levels of the retina and participate, along with bipolar cells, in light-induced generation of the ERG b-wave (Zueva, M. V., 2002). However, Müller cells do not perceive a light rhythm with a frequency higher than 2-4 Hz (Byzov, A. L., 1979). Therefore, whereas during short single stimuli the b-wave reflects the activity of neurons mediated by the buffer properties of the Müller glial cells, during rhythmic stimulation Müller cells do not contribute to the retinal response and the recorded ERG is a purely neuronal potential. Differences in the nature of generation of the ERG and RERG allow indirect estimation of the functional state of Müller cells in a comparative analysis of these biopotentials (Zueva, M. V. and Tsapenko, I. V., 1992). The parameter for determining the function of Müller cells is the glial index (C_{G})—the ratio of the amplitude of the b-wave of the ERG to the amplitude of the RERG at a flicker rate of 8-12 Hz. Analysis of its changes allows estimation the functional and metabolic activities of Müller cells and the entire neuron-neuroglia system in the retina.

Since in the majority of cases the b-wave was practically not recorded or was sharply decreased in all observation periods, the values of C_{G} should be viewed as approaching zero, which is a sign of significant suppression of Müller cell activity and the development of retinal proliferative gliosis, which correlates with clinical observations.

In those cases where C_{G} could be calculated, the dynamics of its values reflects characteristic changes in glial-neuronal interactions in the retina: in the experiment with NSPC, C_{G} after 1 week decreased to 2 rel. units (i.e., Müller cell activity was temporarily suppressed) with its subsequent increase to supernormal values—4.6 units. This increase in C_{G} is indirect confirmation of the activation of Müller cell function and restoration of glial-neural interactions in the retina. In control eyes with kainate retinopathy, 1 week after the experiment either a marked suppression or marked activation of the metabolism in Müller glia was found with an increase in C_{G} to 8-10 units. Such an increase in C_{G} reflects the intensity of compensatory and adaptive mechanisms and in various pathological processes is often evidence of the development of retinal ischemia and hypoxia.

The results of electrophysiological studies performed with the model of kainate retinopathy and NSPC transplantation in early periods are presented in Tables 3 and 4.

TABLE 3
Changes in the amplitude of the ERG and RERG waves in kainate retinopathy
(M ± m) (control group)

| | Before | | | | |
|---|---|---|---|---|---|
| Index | retinopathy | 1 day | 7-8 days | 3 weeks | 1 month |
| ERG a-wave | 38.3 ± 4.4 | 25.5 ± 9.3 | 36.5 ± 10.0 | 26.25 ± 13.9 | 39.9 ± 20.0 |
| ERG b-wave | 140.0 + 16.7 | 0 and 15.5 ± 9.9 | 0 and 49.0 ± 15.4 | 0 and 57.0 ± 9.6 | 0 and 49.5 ± 11.0 |
| C_{G} | 2.3 | 3.90 | 9.60 | 4.10 | 7.60 |
| RERG 12 Hz | 60.0 + 12.3 | 4.0 ± 2.2 | 5.1 ± 2.6 | 14.0 ± 12.0 | 6.5 ± 3.6 |
| RERG 32 Hz | 12.3 ± 2.5 | 0.7 ± 1.2 | 2.0 ± 1.3 | 2.0 ± 0.9 | 1.2 ± 1.0 |

TABLE 4
Changes in the amplitude of the ERG and RERG waves in kainate retinopathy
and transplantation of neural stem cells (M ± m) (experimental group)

| Index | Before retinopathy | 1 day | 7-8 days | 3 weeks | 1 month |
|---|---|---|---|---|---|
| ERG a- | 28.5 ± 5.4 | 22.3 ± 7.7 | 21.7 ± 6.9 | 31.8 ± 16.2 | 6.8 ± 3.4 and 32.0 |
| wave | | | | | |
| ERG b- | 135.0 ± 12.9 | 55.0 ± 14.7 | 57.0 ± 20.0 | 138.0 ± 18.2 | 128.0 ± 18.0 and |
| wave | | | | | 58.0 |
| C_{G} | 2.6 | 4.0 | 2.0 | 4.6 | 6.8 and 4.6 |
| RERG | 52.3 ± 8.8 | 13.7 ± 8.7 | 28.8 ± 11.0 | 29.9 ± 10.0 | 28.0 ± 9.9 |
| 12 Hz | | | | | 8.5 ± 2.0 |
| RERG | 8.2 ± 3.0 | 1.65 ± 1.5 | 3.75 ± 3.0 | 6.55 ± 2.4 | 5.8 ± 3.1 |
| 32 Hz | | | | | |

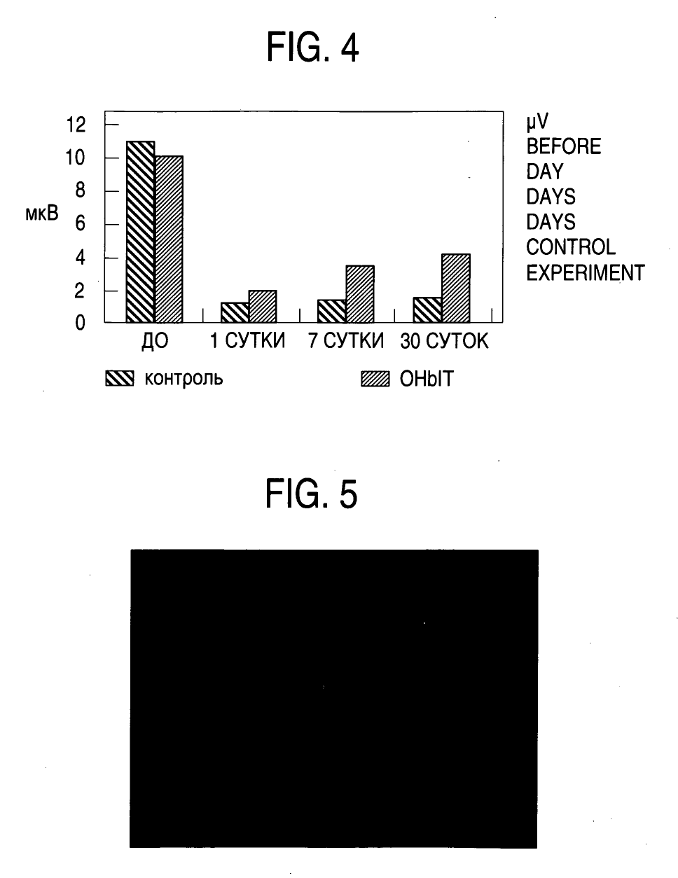

In the series with late administration of NSPC, 5 days after the modeling of retinopathy, the results of electrophysiological studies were not analyzed statistically because of the small amount of experimental material. In view of this, the experimental data obtained thus far do not allow us to see any regularities, although the following tendencies can be identified. In spite of the gross damage to second- and third-order neurons developing at 5 days after injection of kainic acid, partial restoration of the ERG b-wave and an increase in the low-frequency RERG occurred in two cases in the experimental eyes; this indicates some restoration of the rod bipolars by the 30th day of observation (FIG. 3). Possibly, this fact can explain the proliferation and differentiation of transplanted cells with their subsequent replacement of dead neurons. Such a hypothesis requires further investigation with the immunohistochemical studies.

In the series with retrobulbar administration of cells, the nature of changes in retinal electrogenesis in both eyes indicates that the introduction of NSPC promotes the best preservation of the high-frequency ERG (and bipolar cells of the cone system of the retina) and the b-wave of the Ganzfeld ERG (and Müller glial cells). In the control after 1 week and 1 month, a “minus” negative ERG was recorded with a b-wave amplitude below the isoelectric line, amounting to 16.6 and 18:8% of the initial values, respectively (FIG. 3). In the experimental eyes of rabbits with NSPC after injection of kainic acid, with the exception of one case, the b-wave did not disappear completely, but a “plus” negative ERG was recorded with a b-wave amplitude above the response isoelectric line. After a week, some restoration of the b-wave was observed, on average 36.4%, and in some animals up to 50% of the initial value. However, the additional positive effect revealed after 1 week of observation was not preserved upon further observation, and after 1 month the b-wave amplitude decreased almost to the values found 1 day after the injection: to 23.9% of the initial values (FIG. 3).

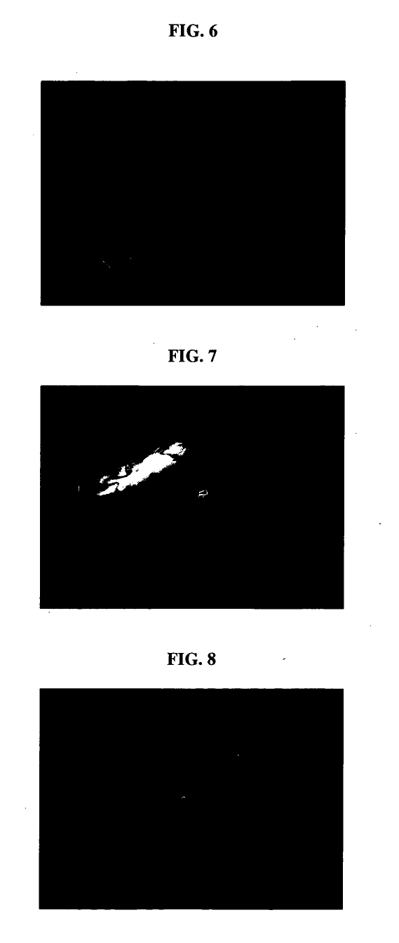

In the control eyes, the amplitude of the RERG to flickers with a rate of 32 Hz increased after 7 days from 11% of the initial values to 11.8% (NS) and by the end of the observation period reached only 12.7% of the initial values. The amplitude of low-frequency RERG at 12 Hz after a week increased to a greater degree, from 11% in the first days to 20.7%, but decreased somewhat 1 month after the injection of kainic acid; the group average was 17.1% (p<0.5) (FIG. 4).

In the experiment, RERGs at 12 and 32 Hz 1 day after the injection amounted to 10.5 and 18% of the initial values, respectively, which did not differ significantly from the reaction of the control eyes. Differences in retinal electrogenesis of the control eyes and the eyes with retrobulbarly introduced SC also appeared at later periods of observation. After 1 week and 1 month of the experiment, the amplitudes of RERGs were 23.4 and 24.9% of the initial amplitudes for a flicker rate of 12 Hz and 33 and 40% for 32 Hz, respectively (the difference in values between the control and experiment was significant at p<0.05) (FIG. 4).

The glial index in the control eyes 1 day after injection of kainic acid tended to zero as a result of the absence of the b-wave. At 1 week, its values amounted to 2.0 rel. units, that is, were significantly decreased; this reflected the risk of development of retinal proliferative gliosis. After 1 month of observation, the average C_{G} for the control eyes was 2.74, but in one rabbit it decreased to 1.5 rel. units; this was consistent with the development of massive retinal gliosis, which was confirmed in the animal upon opthalmoscopic examination.

Our analysis of these data suggested that the retrobulbar administration of neural stem cells exerts a positive effect on the electrogenesis of the rabbit retina in kainate retinopathy. However, this effect is less strongly pronounced than after intravitreal transplantation of NSPC. The best result was obtained for an increase in the functional activity of Miller cells and the retinal cone system.

### CONCLUSIONS

1. A method for NSPC transplantation into the rabbit eye with models of laser damage to the retina and kainate retinopathy was developed for the first time.
2. It was established that NSPC transplantation stimulates the reparative regeneration in the retina when its structural organization is disrupted by laser trauma. The effect is clinically evident in the accelerated pigmentation of the burn defect and morphologically, in the formation of more fragile chorioretinal adhesion.
3. It was proven in immunohistochemical studies that NSPC transplanted into the eye are capable of surviving for at least 1 month and migrating to the area of damage along the retina and choroid; during this process, individual cells retain the status of stem cells.
4. For the first time a stable and easily reproducible model of toxic damage to the rabbit retina—kainate retinopathy—was created, which was manifested electrophysiologically as the induced death of bipolar and Müller cells in the retinal tissue. This model of retinopathy can be used in experimental opthalmology.
5. It was established that transplantation of NSPC decreases proliferative changes in the ocular fundus in kainate retinopathy.
6. It was shown that intravitreal transplantation of NSPC in kainate retinopathy exerts a positive neuroprotective effect on the functional activity of the retina both in the early periods and, in part, in later stages (after the development of gross damage to retinal structures). Retrobulbar administration of NSPC promotes the best preservation of retinal bipolar cells and Müller glial cells.
7. The developed method and the methods and doses of NSPC transplantation, as well as the positive effect obtained with their use, make possible the clinical use of this method in diseases of the posterior section of the eye.

### Example 2

### The Effect of Olfactory Ensheathing Cell Transplantation into the Suprachoroid Space on Recovery Of Retina after Laser Damage

### Materials and Methods

Chinchilla rabbits (body mass 2.5 to 3.0 kg, n=15) were used throughout the study. Two types of laser damage were induced (see the Table 5)

TABLE 5

| | Experimental group | |
|---|---|---|
| | Group 1 | Group 2 |
| Extent of laser | Small laser burns (no | Coagulation of blood vessels |
| damage | deliberate attempts | (produced large areas of |
| | were made to target | ischemic damage) |
| | blood vessels) | |
| Laser power | 300 milliWatt | 600-800 milliWatt |
| Focused beam | 200 micron | 300 micron |
| spot size | | |
| Laser pulse | 100 millisecond | 100 millisecond |
| duration | | |
| Number of burn | 300 spots per eye | 100 spots per eye |
| spots | | |
| Spots location | Lower quadrants of the | Retinal blood vessels |
| | fundus | were coagulated with the laser |
| | | along their entire length. Zone |
| | | of coagulation was inside a |
| | | circle centered around optic |
| | | disk. Circle radius was half |
| | | the equatorial radius of the |
| | | eye. |
| Number of | 5 rabbits | 10 rabbits |
| animals | | |
| Injection site | supra-choroid space | supra-choroid space |
| Number of cells | 500,000 cells/20 μl | 500,000 cells/20 μl |
| (*) per injection | | |
| Cells were | Right Eye | Right Eye |
| injected into | | |
| Negative control | Left eye (20 μl of | Left eye (20 μl of saline |
| | saline alone) | alone) |

### Eye Morphology

Eye morphology was studied by using biomicroscopy (slit-lamp exam) and opthalmoscopy (examination of the fundus). Light microscopy was used to evaluate histopathology of the retina. Eyes were harvested 7, 14, 30 and 40 days after injection of Olfactory Ensheathing Cells.

### Electroretinography

ElectroRetinoGraphy was performed according to the standards of the International Society for Clinical Electrophysiology of Vision (http://www.iscev.org/standards/pdfs/erg-standard-2004.pdf) and was recorded using a contact lens electrode. The electrode was placed on the cornea after topical instillation of anesthetic (0.5% tetracaine hydrochloride). The pupils were dilated with 1% Midriacil® to reduce variation caused by pupil contractions. Reference electrode and ground electrode were implanted under the scalp. Corneal Ganzfeld ERG's were recorded using a stimulus of 5 cd×sec×m̂(−2) (candela-seconds per meter squared). Rhythmic stimulation was done at 12 and 32 Hz. Electroretinography was recorded 25, 30 and 40 days after laser damage. In a separate series of experiments, electroretinography was recorded before, and on day 10, day 18 after laser damage. Stimulus strength varied across 1.2 log range (10̂1.2=16-fold). Flash light intensity varied between 1.8 and 28.5 candela×sec×m̂(−2).

### Group 1 (Small Laser Burn Spots)

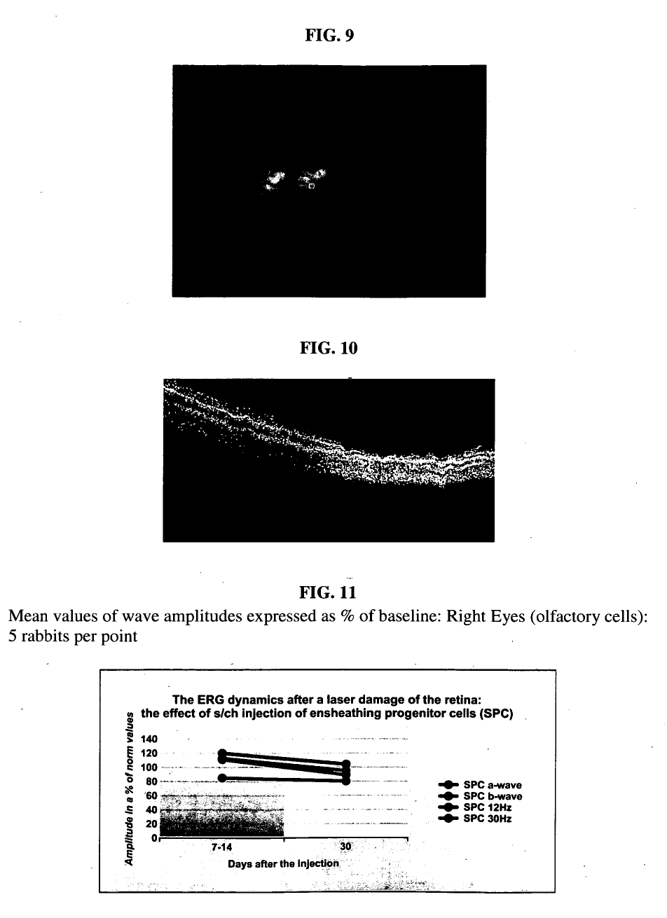

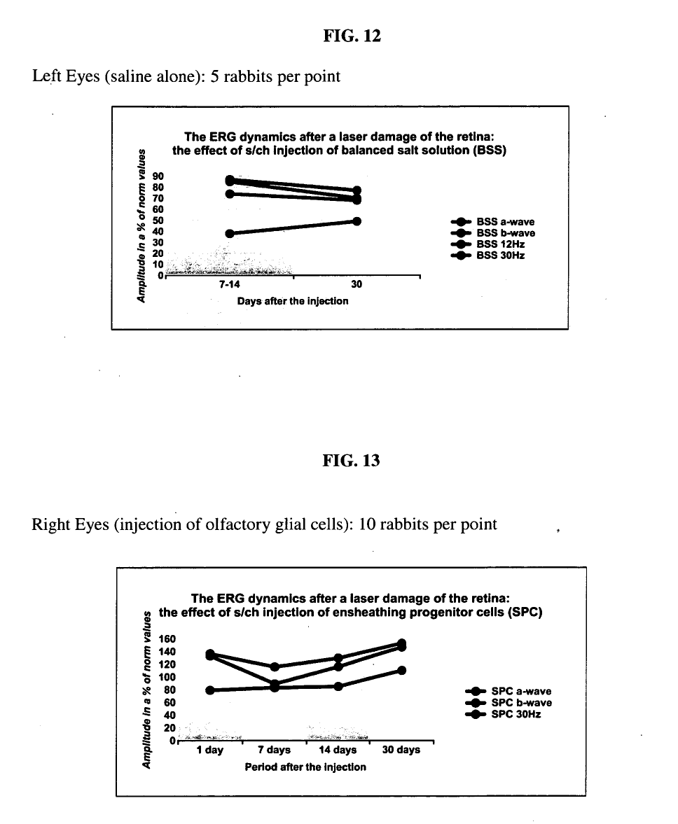

Superficial and inner blood vessels (scleral) were dilated the next day after laser damage. Vascular reaction subsided after 3 days. Slit-lamp exam showed transparent vitreous humor, clear lens and cornea. Iris was intact. Retinal blood vessels were dilated. Laser burn spots were swollen. On day 3, vascular reaction began to calm down. Burn spots appeared darker due to proliferation of pigment cells. Pigment cells first appeared on periphery and then spread to the center of the burn spots. On day 5-7, vascular reaction and edema disappeared. Colonization of burn spots with pigment cells was complete by day 7. Pigmentation of burn spots reached a plateau by day 30. See FIGS. 5-6.

### Group 2 (Blood Vessel Coagulation; Large Ischemic Lesions)

Pronounced constriction of all retinal blood vessels occurred immediately after coagulation. Vasoconstriction was accompanied by almost complete circulation arrest that lasted for 2-5 minutes. Circulation started to recover after 5-10 minutes but vasoconstriction did not go away.

The next day after coagulation, central area of the retina was swollen, optic nerve head boundaries were blurred. Most of the arteries were constricted, some collapsed. Veins had bulges and ampullar dilatations. Peripheral areas of the retina were relatively unaffected.

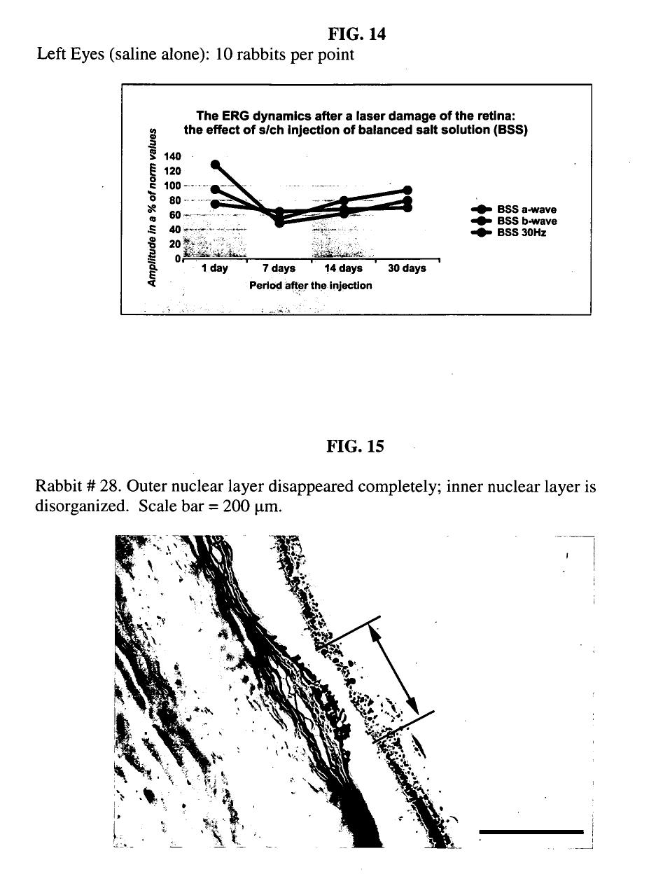

Circulation slowly returned to normal within 1 week. Many arteries remained constricted; there were some occlusion areas. Veins appeared dilated and distorted (loops and coils). Retinal tissue around burnt vessels appeared less swollen compared to first 2-3 days. Two types of lesions were present: small burn spots and large lesions caused by interrupted circulation. Large lesions could be seen not only in the center, but also in the periphery of the fundus. Total number of lesions varied greatly among animals. Several weeks later, collateral blood vessels began to appear. Constricted arteries began to relax. Veins remained dilated and distorted. See FIGS. 7-10.

### ElectroRetinoGraphy

Pilot experiment #1: Two rabbits were given an injection of olfactory ensheathing cells without laser treatment. Half million cells in 20μ saline had been injected into the suprachoroid space. One week after injection, ERG data decreased slightly (10-15% below baseline). There was no difference between the right eye (cells) and left eye (saline alone). Histological exam showed no tissue damage at sites of injection.

Pilot experiment #2: Twenty rabbits received 2 types of laser treatment but no cells. Small laser burns were produced in a group of 10 rabbits; blood vessel coagulation was performed in a group of another 10 rabbits. In the first group (small burns), a- and b-wave amplitudes (in response to single flash ERG?) were elevated 7 days after treatment. ERG amplitudes slowly returned to normal (after 7 days?). Flicker ERG amplitudes were reduced throughout the observation period (up to 1 month.)

TABLE 6

| | | | |
|---|---|---|---|
| Time after laser damage | Single flash ERG | 12 Hz flicker ERG | 32 Hz flicker ERG |

| | | | |
|---|---|---|---|
| a-wave | right after coagulation | 50-60% below baseline | |
| b-wave | right after coagulation | 25-30% below baseline | |
| a-wave | 7 days after damage | | decreased |
| b-wave | 7 days after damage | | decreased |
| a-wave | 14 days after damage | | 40% below baseline |
| b-wave | 14 days after damage | | 40% below baseline |
| a-wave | 30 days after damage | | still below baseline |
| b-wave | 30 days after damage | | still below baseline |

In second group of animals (ischemia caused by vessel coagulation), single flash ERG data showed an increase in a-wave amplitudes that were 20-40% above baseline during first 2-3 days after coagulation. b-wave amplitudes depended on the size of ischemic damage. If ischemic area was large, b-waves fell below baseline. If ischemic area was small, b-waves elevated 20-60% above baseline. Flicker ERG was always below baseline and never completely recovered during 1 month observation.

Group 1—Treatment with Olfactory Ensheathing Cells

TABLE 7

| Time | | | | b-wave |
|---|---|---|---|---|
| after damage | ERG type | Treatment | a-wave | 1-2 weeks |
| 1-2 weeks | Single flash | olfactory glia (R) | 11% above baseline | 13% above baseline |
| 1-2 weeks | Single flash | saline alone (L) | 38-85% below baseline | 38-85% below baseline |
| 1-2 weeks | 12 Hz flicker | olfactory glia (R) | 20-30% above baseline | 20-30% above baseline |
| 1-2 weeks | 12 Hz flicker | saline alone (L) | 20-30% above baseline | 20-30% above baseline |
| 1-2 weeks | 32 Hz flicker | olfactory glia (R) | slightly below baseline | slightly below baseline |
| 1-2 weeks | 32 Hz flicker | saline alone (L) | slightly below baseline | slightly below baseline |

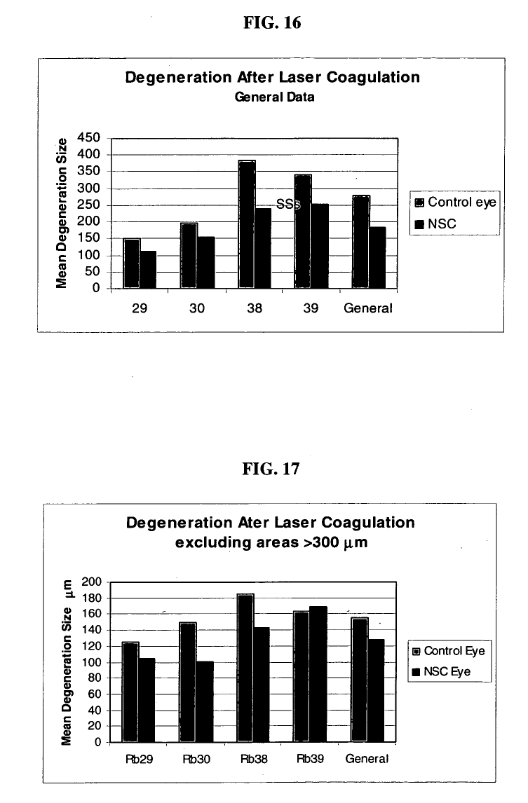

After 2 weeks, 2 rabbits demonstrated slight depression of waves in response to single flash and flicker stimulation. That depression was less pronounced in right eyes (olfactory cells) compared to left eyes (saline). After 4 weeks, electric responses in 3 out of 5 rabbits demonstrated a “positive trend toward recovery”. Electric responses were highly variable at early stages after cell injection, and differences between the left and right eye did not reach statistical significance (P>0.05). However, after 4 weeks, the authors had detected differences that were significant at P level <0.05. See FIGS. 11-12.

Group 2—Treatment with Olfactory Ensheathing Cells

Ten rabbits were used. Retinal vessels of 1^{st}, 2^{nd}, 3^{rd} order were coagulated. (“The major retinal arterioles that arise from the optic disc are classified as the first-order vessels (I), and subsequent branches are defined as second (II) and third-order (III) vessels”). The next day after vessel coagulation, electric responses declined below baseline in 3 out of 10 rabbits (both eyes). Photoreceptor function (a-waves) had suffered the most (45-65% drop below baseline).

In 7 out of 10 rabbits, electric response of the retina increased (“compensatory hyper reaction”). Averaged a-wave amplitudes were 30% above baseline. There were no differences between control left eye and cell-treated right eye. Most likely, increased electric response occurred because of occlusion of 1^{st} order arterioles and subsequent ischemia of the retina. b-wave amplitudes depended on the size of ischemic damage. If ischemic area was large, b-wave amplitudes declined. If ischemic area was small, b-waves rose 35-55% above baseline. During first 1-5 days post-treatment, there were no differences between control left eye and cell-treated right eye.

TABLE 8

| | | Right eye (olfactory glia) → FIG. |
|---|---|---|
| | Left eye (saline) → FIG. 2A | 2B |
| day 1 | a- and b-waves increased above baseline | a- and b-waves increased above baseline |
| day 7 | In 4 out of 10 rabbits, elevated a- and b-waves | Elevated a-waves declined 11% below pre- |
| | returned to pre-treatment level. In 6 out of 10 | treatment level. b-waves stayed elevated |
| | rabbits, elevated a- and b-waves declined below | above baseline. |
| | pre-treatment level. | |
| day | Average a-wave amplitude was 20% below | In 6 out of 10 rabbits, a- and b-waves went |
| 14, | baseline. | 16-70% above baseline. |
| day 30 | Average b-wave amplitude was 6% below | In 4 out of 10 rabbits, b-waves went 56% |
| | baseline. | above baseline (that probably reflected |
| | In 7 out of 10 animals, response to 30 Hz | activation of Muller glial cells). Response to |
| | flickers was 25-40% below baseline. In 3 out of | 30 Hz flickers returned to normal in 8 out of |
| | 10 animals, response to 30 Hz flickers was 10-15% | 10 rabbits. |
| | below baseline in all animals. | |
| | ERG data and morphologic exams supported | |
| | conclusion about accumulation of dystrophic | |
| | areas in the retina. | |

FIG. 13 demonstrates mean values of wave amplitudes expressed as % of baseline. NB. Differences between the right eye (olfactory cells) (FIG. 13) and the left eye (saline) (FIG. 14) were significant (P<0.05) at day 1 and day 30. At day 7 and day 14, individual variation between animals blurred the difference, but the trend was clear (the right eye responded to light better than left eye).

### Erg Data: Concluding Remarks

(1) Olfactory glial cells injected into suprachoroid space protected retina from ischemic damage during the first day after laser coagulation.
(2) Olfactory glial cells improved recovery of retinal cells, particularly Muller glial cells (see b-waves on day 30).

### Histological Study of Laser Damaged Retina.

Eye preparation for histological examination. Rabbits were euthanized. Without delay, animals were intra-cardially perfused with ice-cold isotonic saline followed by 4% paraformaldehyde solution in phosphate buffered saline. The eyes were removed at the end of perfusion. The lens was removed through a cut in the cornea. Eyes were kept at 4° C. in 4% paraformaldehyde for 12-14 hr. Eyes were dehydrated in ethanol and embedded in paraffin. Ten micrometer thick serial sections were made across vertical plane. Every 50^{th} section was mounted and stained with Giemsa stain. Every 100^{th} section was subjected to morphometric analysis. Laser burn spots containing no photoreceptor cells were measured by using “Image Tool” software.

Human mesenchymal cells were cultured with Bromodeoxyuridine (BrdU) to label their DNA. BrdU inside human cells was detected by immunohistochemical method. Briefly, sections were deparaffinized and placed into 3% H₂O₂ in PBS+10% methanol for 30 min to block endogenous peroxidase. DNA was denatured in 2 M HCl for 45 min. HCl was neutralized by dipping slides into 0.1 M sodium borate, pH 8.5 for 10 min. Slides were washed in PBS for 10 min. Sections were incubated with mouse anti-BrdU antibody diluted 1:10 (ICN Biomedicals Inc., www.icnbiomed.com) for 12-15 hr at 4° C. Sections were washed 3 times in PBS and incubated with goat anti-mouse IgG labeled with biotin (diluted 1:100). After 3 washes, biotinylated antibody was detected by Avidin-Peroxidase (ABC complex from Vector Labs). Peroxidase activity was detected by DAB method. Some slides were stained with goat anti-mouse IgG labeled with Texas red fluorescent dye.

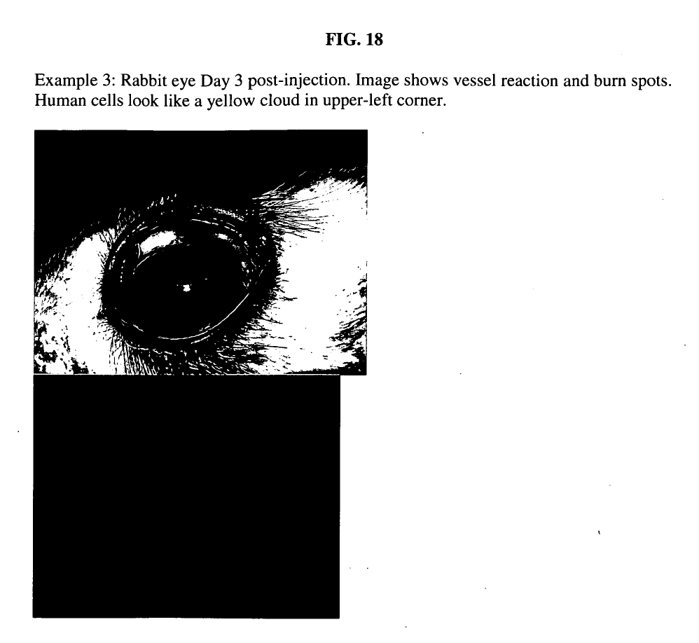

The authors found no “atypically located cells” (olfactory glial cells?) 3-4 weeks after injection. There was a partially closed needle puncture hole at the injection site. Retinal histology was normal around the puncture hole. Injected cells were not found. Retinal layers were damaged in places where laser beam hit the retina: outer nuclear layer had disappeared completely; inner nuclear layer looked disorganized and compacted in many places (see FIG. 15).

There were 2 types of lesions caused by the laser. Small burn spots were 50 to 300 micron in size. There were also larger secondary lesions that developed due to ischemia (when laser beam hits a small artery, large area of the retina suffers because of interrupted blood flow→secondary lesion). The number of large ischemic lesions varied widely between animals.

Areas, where photoreceptors were completely destroyed, were measured by using “Image Tool” software (20-30 serial sections were analyzed for every eye).

Despite large size variation, average size of lesions in the right eye (olfactory glia) was lower than average size of lesions in the left eye (saline alone).

In order to reduce variation caused by ischemia, lesions larger than 300 μm were exluded from analysis (see Table 9).

TABLE 9
Averaged size of damaged areas (small spots + ischemic
areas larger than 300 μm)

| | Left eye | Right eye | |
|---|---|---|---|
| | saline alone | olfactory glial cells | P value |
| Rabbit #29 | 152 | 111 | P < 0.05 |
| Rabbit #30 | 197 | 156 | |
| Rabbit #38 | 384 | 241 | P < 0.05 |
| Rabbit #39 | 341 | 252 | |
| Mean values | 281 | 184 | P < 0.05 |

TABLE 10
Averaged size of damaged areas (only areas smaller than
300 μm were counted)

| | Left eye | Right eye | |
|---|---|---|---|
| | saline alone | olfactory glial cells | P value |
| Rabbit #29 | 125 | 105 | P < 0.05 |
| Rabbit #30 | 150 | 101 | P < 0.05 |
| Rabbit #38 | 185 | 142 | P < 0.05 |
| Rabbit #39 | 164 | 169 | |
| Mean values | 155 | 128 | P < 0.05 |

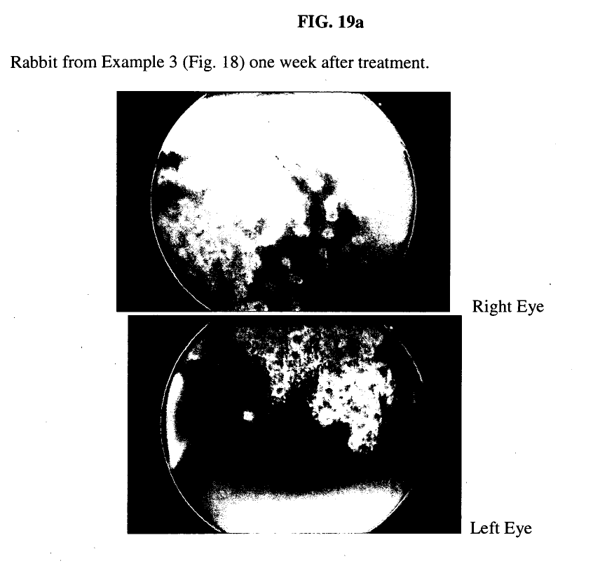

FIG. 16 duplicates data shown in Table 9 (NSC=olfactory glial cells)

FIG. 17 duplicates data shown in Table 10 (NSC=olfactory glial cells)

Histological picture differed significantly from local laser burn spots. Areas of ischemic damage were much larger (300 μm-1,000 μm). Retinal layers disappeared completely. Thickness of the retina was reduced. Areas varied in size. There were no differences between the left eye (saline alone control) and right eye (olfactory glial cells).

### Example 3

### The Effect of Human Mesenchymal Cell Transplantation in the Treatment of Retina Laser Damage

### Materials and Methods

Chinchilla rabbits (body mass 2.5 to 3.0 kg, n=37) were used throughout the study. Laser-induced retinal damage was used as a model to simulate exudative macular degeneration in humans. Laser photocoagulation (200 μm spot size, 100 millisecond duration, 300 mW laser power ______ nm wavelength) was used to generate 300 laser spots in the lower quadrant(s) of each eye.

TABLE 11
Animals were divided into 3 experimental groups:

| | Exp. | Exp. | Exp. |
|---|---|---|---|
| | Group 1 | Group 2 | Group 3 |
| Number of human | 300,000 cells | 300,000 cells | 300,000 cells |
| mesenchymal cells | | | |
| injected | | | |
| injection site | into the | into the | into the |
| | vitreous cavity | suprachoroid | suprachoroid |
| | | space | space (*) |
| injection volume | 100 microL | 20 microL | 20 microL |
| Time between human | 30 days | 30-40 days | 10-18 days |
| cell transplantation | | | |
| and animal euthanasia | | | |
| Measuring equipment | RetinoGraf | RetinoGraf | Mjölner** |

**ERG system Mjolner → http://www.globaleyeprogram.com/htm/mjolner.htm
(*) The suprachoroid is a potential space lying between the inner scleral wall and the apposing choroid coat
http://www.freepatentsonline.com/5443505.html)

TABLE 12

| | Control eye | Treated eye |
|---|---|---|
| Left/Right | ? left eye | ? right eye |
| | (oculus sinister) | (oculus dexter) |
| Laser | 100 millisecond, | 100 millisecond, 300 mW, |
| damage | 300 mW, 200 μm spot, 300 | 200 μm spot, 300 spots |
| | spots per eye | per eye |
| Human cells | 0.95% NaCl saline alone | 300,000 cells in 0.95% NaCl |
| | | saline |

Eye morphology was observed by using biomicroscopy (slit-lamp exam) and opthalmoscopy (examination of the fundus). Light microscopy was used to evaluate eye histopathology. Eyes were obtained on day 7, day 30 and day 40 after injection of human mesenchymal cells.

ElectroRetinoGraphy was performed according to the standards of the International Society for Clinical Electrophysiology of Vision (http://www.iscev.org/standards/pdfs/erg-standard-2004.pdf) and was recorded using a contact lens electrode. ElectroRetinoGraphy was recorded using a contact lens electrode. The electrode was placed on the cornea after topical instillation of anesthetic (0.5% tetracaine hydrochloride). The pupils were dilated with 1% Midriacil® to reduce variation caused by pupil contractions. Reference electrode and ground electrode were implanted under the scalp. Corneal Ganzfeld ERG's were recorded using a stimulus of 5 cd×sec×m̂(−2) (candela-seconds per meter squared). Rhythmic stimulation was done at 12 and 32 Hz. Electroretinography was recorded 25, 30 and 40 days after laser damage. In a separate series of experiments, electroretinography was recorded before, and on day 10, day 18 after laser damage. Stimulus strength varied across 1.2 log range (10̂1.2=16-fold). Flash light intensity varied between 1.8 and 28.5 candela×sec×m̂(−2).

### Experimental Group #1

Human mesenchymal cells were injected into the vitreous cavity (300,000 cells in 100 microL). Ten animals were used. Cells were injected into the right eye; the left eye served as a negative control.

Results: Superficial and inner blood vessels were dilated the next day after laser damage. Vascular reaction subsided after 3-5 days. Slit-lamp exam showed transparent vitreous humor, clear lens and cornea. Iris was intact. The pupillary reflex was normal. Examination of the fundus showed that, the next day after injection, the majority of human mesenchymal cells could be seen suspended in vitreous humor. Injected cells formed a fluffy mass (flocculate). Retinal blood vessels were dilated. Laser burn spots were swollen. On day 3, vascular reaction began to calm down. Burn spots appeared darker due to proliferation of pigment cells. Pigment cells first appeared on periphery and then spread to the center of the burn spots. On day 5-7, vascular reaction and edema disappeared. Burn spots appeared darker. Rate of pigmentation was similar in treated eye and negative control eye.

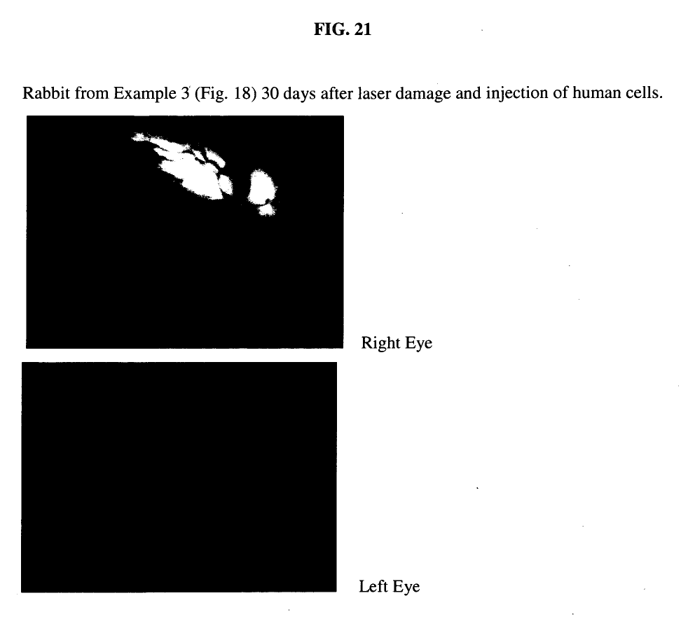

One and two weeks after treatment, slit-lamp exam revealed no signs of inflammation in both eyes; injected human cells could be seen suspended in vitreous humor. Examination of the fundus revealed that the majority of injected cells stayed as one big cluster. The size of the cluster began to shrink. Optic disk was pale-pink in color. Retinal vessels were not dilated. Accumulation of pigment cells inside burn spots was completed (see FIG. 19).

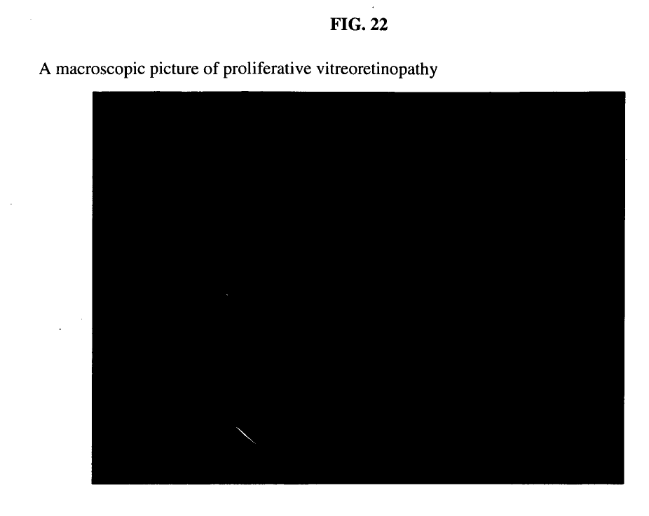

Two to three weeks after treatment, slit-lamp exam revealed no signs of inflammation in both eyes (see FIG. 20). Injected human cells could be seen in small clusters. The clusters were floating in vitreous liquor at the bottom of the eye near the equator. Burn spots appeared to be more pigmented.

ElectroRetinoGraphy

TABLE 13

| | Time after laser damage | Single flash ERG | 12 Hz flicker ERG | 32 Hz flicker ERG |
|---|---|---|---|---|
| a-wave | 2 days after damage | 43% below normal | 91% below normal | 30%-50% above normal |
| b-wave | 2 days after damage | 80% below normal | 99% below normal | normal |
| a-wave | 25 days after damage | ? | ? | ? |
| b-wave | 25 days after damage | 75% below normal | 65% below normal | 62% below normal |

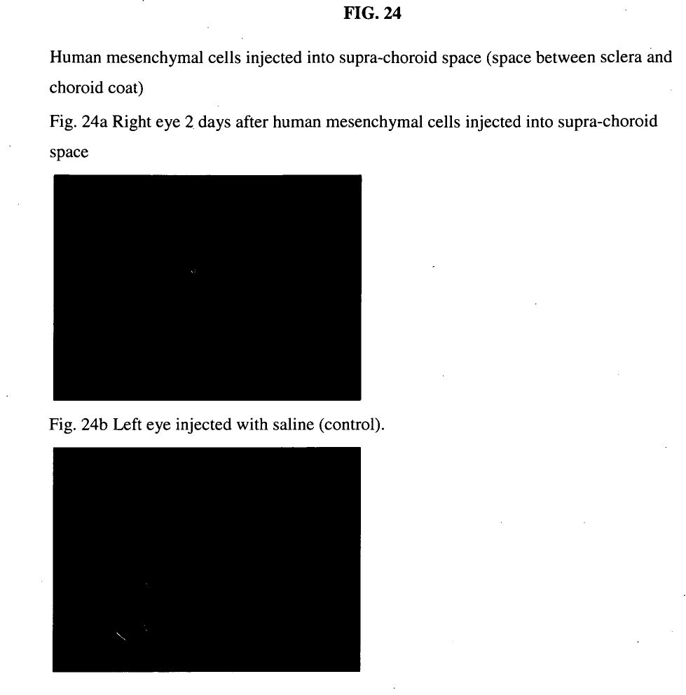

25 days after laser damage, electric activity of retina continued to decrease. Histological examination confirmed diagnosis of Proliferative VitreoRetinopathy, see FIG. 22.

After laser damage, glial index decreased from its normal value of 2.8 to 1.9.

ElectroRetinoGram data 25 days after laser damage.
Three columns on the left→Treatment: eye injected with human mesenchymal cells.
   Grey column→normal value (100%)
   Green column→a-wave amplitudes (≈40%)
   Blue column→b-wave amplitudes (≈70%)
Three columns on the right→Control: eye injected with saline.
   Grey column→normal value (100%)
   Green column→a-wave amplitudes (≈38%)
   Blue column→b-wave amplitudes (≈65%)

### Experimental Group #2

12 animals were used. Cells were injected into the right eye; the left eye served as a negative control.

Results: Superficial and inner blood vessels were dilated on the next day after laser damage. Vascular reaction subsided after 3 days. Slit-lamp exam showed transparent vitreous humor, clear lens and cornea. Iris was intact. The pupillary reflex was normal. Retinal blood vessels were dilated. Laser burn spots were swollen. On day 3, vascular reaction began to calm down. Burn spots appeared darker due to proliferation of pigment cells. Pigment cells first appeared on periphery and then spread to the center of the burn spots. On day 5-7, vascular reaction and edema disappeared. Burn spots appeared darker. Rate of pigmentation was similar in treated eye and negative control eye.

ElectroRetinoGraphy was measured 10, 18, 25, 30 and 40 days after laser damage.

TABLE 14

| Time | | | | | |
|---|---|---|---|---|---|
| after | | | | 12 Hz | |
| laser | | | Single flash | flicker | 32 Hz flicker |
| damage | Treatment | | ERG | ERG | ERG |
| 10-18 | control eye | a- | pronounced | ? | pronounced |
| days | | wave | decrease | | decrease |
| 10-18 | control eye | b- | close to normal | ? | ? |
| days | | wave | | | |
| 10-18 | mesenchymal | a- | slightly better | ? | slightly |
| days | cells | wave | than control* | | better |
| | | | | | than control |
| 10-18 | mesenchymal | b- | close to normal | ? | ? |
| days | cells | wave | | | |
| 25-40 | control eye | a- | slightly below | slightly | slightly |
| days | | wave | normal | below | below |
| | | | | normal | normal |
| 25-40 | control eye | b- | ? | ? | ? |
| days | | wave | | | |
| 25-40 | mesenchymal | a- | ? | 20-52% | 20-52% |
| days | cells | wave | | above | above |
| | | | | normal | normal |
| 25-40 | mesenchymal | b- | ? | ? | ? |
| days | cells | wave | | | |

There were no statistically significant differences (P>0.05)

30 days after treatment, electrical response 12 Hz stimulation completely recovered; it even rose slightly above normal level. There were no significant differences between treated and sham treated eyes. Eyes injected with human mesenchymal cells demonstrated less pronounced suppression of electric activity as compared to sham-treated eyes.

TABLE 15

| 30 days | Mesenchymal cells | Saline alone |
|---|---|---|
| after treatment | (right eye) | (left eye) |
| a-wave | 61-87% below normal | 52-69% below normal |
| b-wave | 90-93% below normal | 85-86% below normal |

Authors speculated that human mesenchymal cells injected into supra-choroid space caused accelerated recovery of burnt retina.

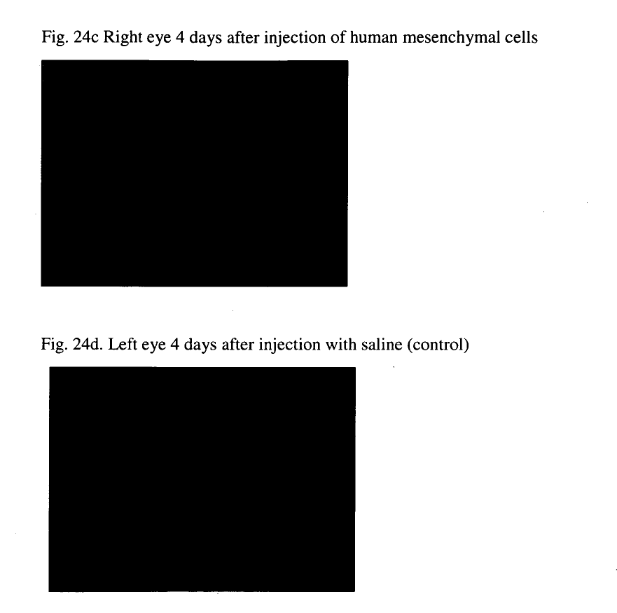

Single flash ERG and 12 Hz flicker ERG data were recorded 25-40 days after laser damage and suprachoroid injection of human mesenchymal cells (FIG. 23).

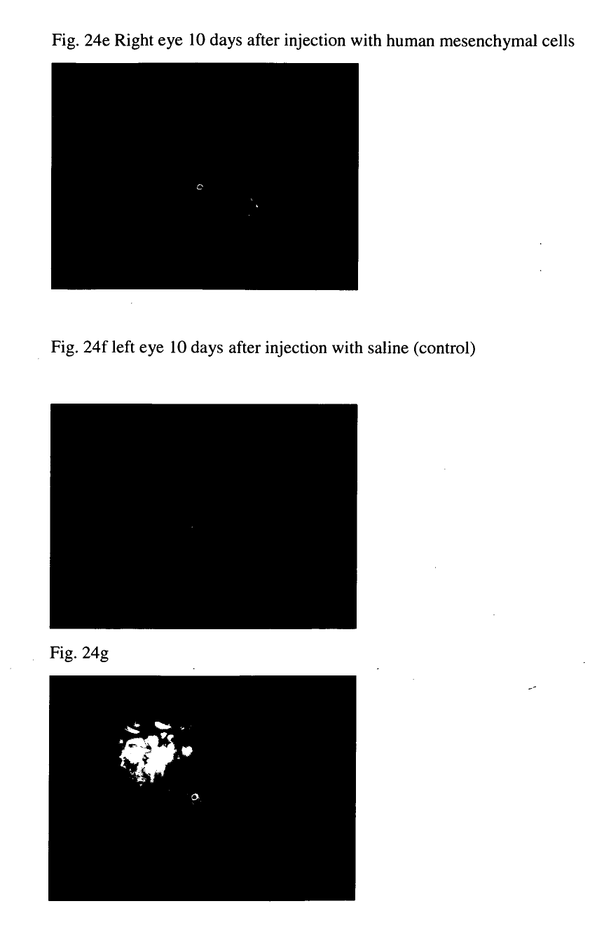

FIG. 24 depicts results from human mesenchymal cells injected into supra-choroid space (space between sclera and choroid coat) two, four and 10 days after injection.

### Experimental Group #3

15 animals were used. Cells were injected into the right eye; the left eye served as a negative control. Electric activity of burnt retina was suppressed after 10 and after 18 days. a-waves amplitudes induced by single flash ERG and 30 Hz flicker ERG declined the most. b-wave amplitudes were close to normal.

Rabbit #32. ERG data for the right eye are shown (eye injected with human mesenchymal cells). Top row→singe flash ERG data. Bottom row→32 Hz flicker ERG data.

Stimulus strength=flash light intensity measured as delta above ambient room light level

TABLE 16

| log N | 10\hat{ }N | candela × sec × meter\hat{ }(−2) |
|---|---|---|
| 0 log | 1 | 1.8 candela × sec × m\hat{ }(−2) |
| 0.3 log | 2 | 3.6 candela × sec × m\hat{ }(−2) |
| 0.6 log | 4 | 7.2 candela × sec × m\hat{ }(−2) |
| 0.9 log | 8 | 14.3 candela × sec × m\hat{ }(−2) |
| 1.2 log | 16 | 28.5 candela × sec × m\hat{ }(−2) |

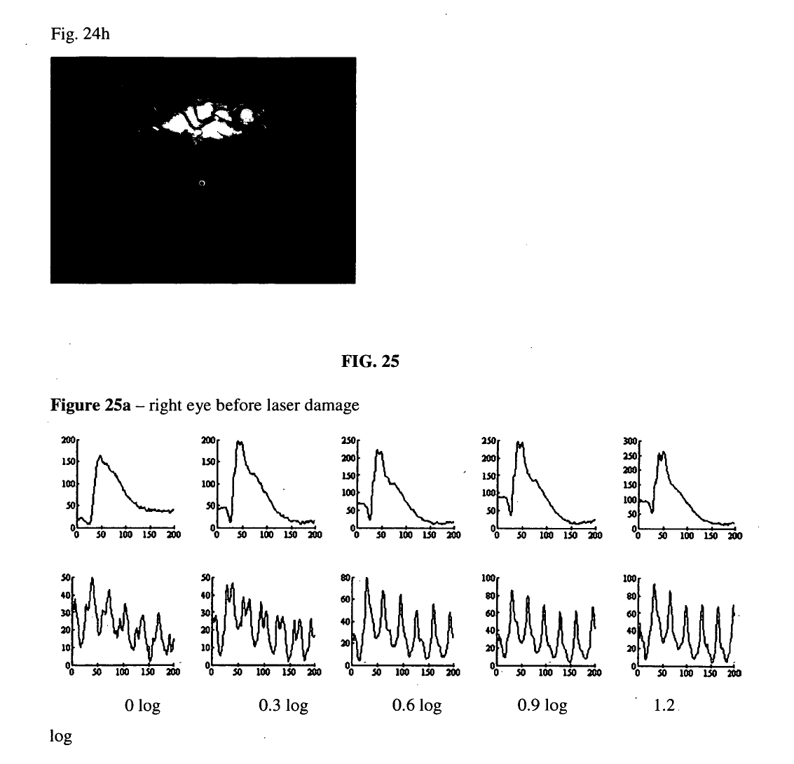

FIGS. 25D-F: Same rabbit as in FIG. 10 (rabbit #32).

ERG data for the left eye are shown (eye injected with saline).
Top row→singe flash ERG data.
Bottom row→32 Hz flicker ERG data.

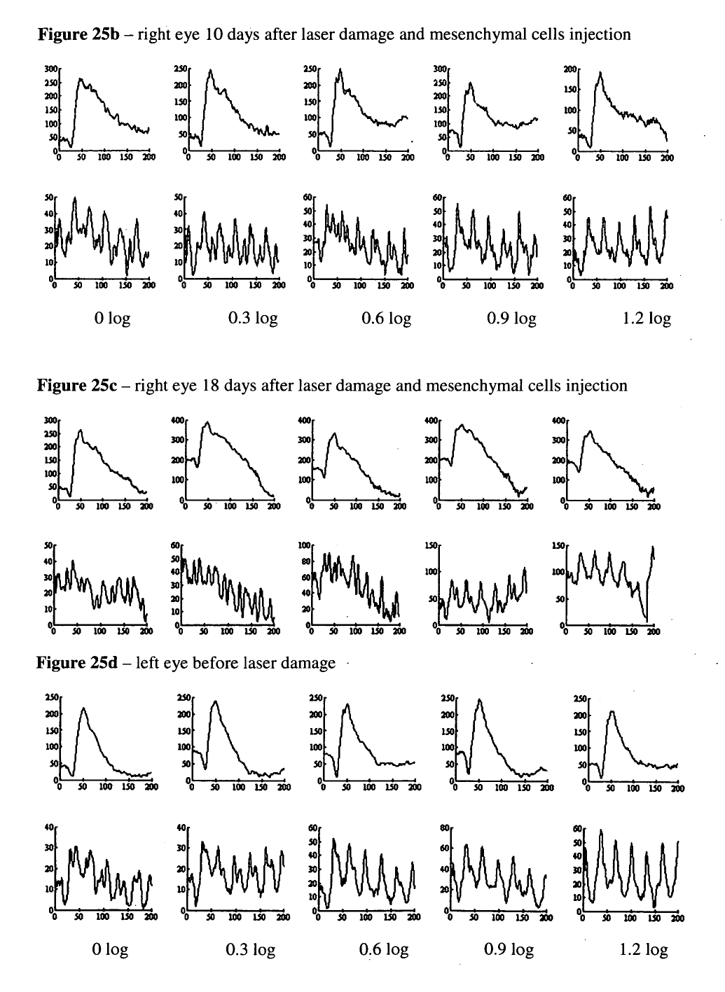

FIGS. 26 and 27 illustrate individual variation of ERG data as well as mean amplitude values. In case of one animal, injection of mesenchymal cells into both eyes caused increase of a-wave amplitudes above baseline. But mean values for the whole group of animals did not change (there was no difference between saline and cells).

FIG. 26 depicts individual variation of ERG data. Two rabbits (3 eyes) data are plotted. 10 days after laser damage+mesenchymal cell injection

FIG. 27 depicts averaged ERG data: 10 days and 18 days after laser damage+mesenchymal cell injection.

### CONCLUSIONS

(1) Injection of human mesenchymal cells into the vitreous cavity did not improve recovery of burnt retina.
(2) 25-30 days after laser damage, injection of human mesenchymal cells into the suprachoroid space slightly improved response of burnt retina to 12 Hz flickers.

TABLE 17
ERG dynamics after laser damage and suprachoroid injection of mesenchymal stem cells.

| | | | | | | | | | | | | 30 Hz flicker ERG | | | | |
|---|---|---|---|---|---|---|---|---|---|---|---|---|---|---|---|---|
| | | Single flash ERG | | | | | | | | | | 0 | 0.3 | 0.6 | 0.9 | 1.2 |
| | | 0 log | | 0.3 log | | 0.6 log | | 0.9 log | | 1.2 log | | log | log | log | log | log |
| Amplitude | | a | b | a | b | a | b | a | b | a | b | Peak-to-peak | | | | |
| Mesenchymal | day 0 | 26 | 269.3 | 43 | 252 | 52 | 237.7 | 56 | 234 | 41 | 215 | 47.7 | 54.7 | 68.7 | 76.7 | 59 |
| cells | day | 22 | 236.7 | 32 | 239.3 | 40 | 232.7 | 49 | 238.3 | 29.3 | 205.7 | 36.7 | 39 | 42.3 | 47.3 | 39.4 |
| (the right | 10 | | | | | | | | | | | | | | | |
| eye) | day | 27 | 268.3 | 34 | 234 | 36 | 227 | 37 | 222 | 45 | 210 | 41.5 | 42 | 55.5 | 65.5 | 62 |
| | 18 | | | | | | | | | | | | | | | |
| Saline | day 0 | 22 | 249 | 42 | 238 | 49 | 242 | 55 | 240 | 44 | 245 | 43 | 56 | 64 | 67 | 51 |
| alone (the | day | 21 | 182 | 30 | 188 | 38 | 203 | 43 | 196 | 25 | 184 | 36 | 44 | 48 | 55 | 40 |
| left eye) | 10 | | | | | | | | | | | | | | | |
| | day | 24 | 241 | 34 | 222 | 34 | 230 | 33.5 | 239 | 44 | 255 | 45 | 51 | 62.5 | 69.5 | 56.8 |
| | 18 | | | | | | | | | | | | | | | |

### Histological Analysis of Laser Damaged Retina

Eye preparation for histological examination. Rabbits were euthanized. Without delay, animals were intracardially perfused with ice-cold isotonic saline followed by 4% paraformaldehyde solution in phosphate buffered saline. The eyes were removed at the end of perfusion. The lens was removed through a cut in the cornea. Eyes were kept at 4° C. in 4% paraformaldehyde for 12-14 hr. Eyes were dehydrated in ethanol and embedded in paraffin. Ten micrometer thick serial sections were made across vertical plane. Every 50^{th} section was mounted and stained with Giemsa stain. Every 100^{th} section was subjected to morphometric analysis. Laser burn spots containing no photoreceptor cells were measured by using “Image Tool” software.

Human mesenchymal cells were cultured with Bromodeoxyuridine (BrdU) to label their DNA. BrdU inside human cells was detected by immunohistochemical method. Briefly, sections were deparaffinized and placed into 3% H₂O₂ in PBS+10% methanol for 30 min to block endogenous peroxidase. DNA was denatured in 2 M HCl for 45 min. HCl was neutralized by dipping slides into 0.1 M sodium borate, pH 8.5 for 10 min. Slides were washed in PBS for 10 min. Sections were incubated with mouse anti-BrdU antibody diluted 1:10 (ICN Biomedicals Inc., www.icnbiomed.com) for 12-15 hr at 4° C. Sections were washed 3 times in PBS and incubated with goat anti-mouse IgG labeled with biotin. After 3 washes, biotinylated antibody was detected by Avidin-Peroxidase. Peroxidase activity was detected by DAB method. Some slides were stained with goat anti-mouse IgG labeled with Texas red fluorescent dye.

Retinal Histology after Mesenchymal Cell Injection into the Vitreous Cavity

24 hr after laser damage, there was strong proliferation of fibroblast-like cells. Optic nerve disk protruded into the vitreous cavity.

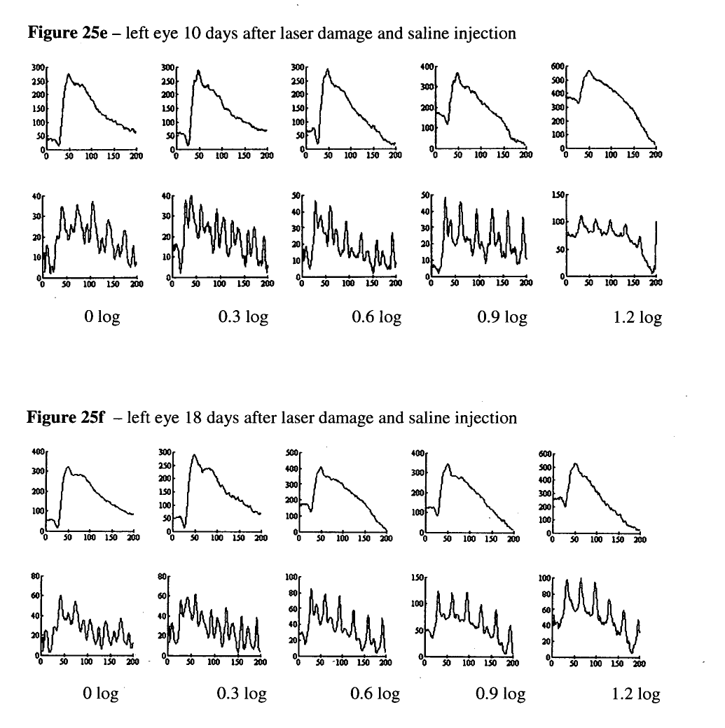

FIG. 28 depicts a microphoto of Rabbit #22 24 hr after laser damage and transplantation of human mesenchymal cells into the vitreous cavity.

FIG. 29 depicts microphoto of Rabbit #22 24 hr after laser damage and transplantation of human mesenchymal cells into the vitreous cavity (FIG. 28) at higher magnification.

Retinal Histology after Mesenchymal Cell Injection into the Supra-Choroid Space

There were 2 types of lesions caused by the laser. Small burn spots were 50 to 300 micron in size. There were also larger secondary lesions that developed due to ischemia (when laser beam hits a small artery, large area of the retina suffers because of interrupted blood flow→secondary lesion). The number of large ischemic lesions varied widely between animals. To reduce this variation and unmask potential beneficial effect of mesenchymal cells, small lesions (less than 300 micron in size) were counted separately.

TABLE 18
Integrated size of all lesions: Small laser burns (<300 micron)
and Large ischemic areas (>300 micron) were combined.

| | Treated eye | Control eye |
|---|---|---|
| N = 6 | mean values | mean values |
| Rabbit #27 | 1.031 mm | 1.399 mm |
| Rabbit #28 | 0.163 mm | 0.151 mm |
| Rabbit #32 | 0.219 mm | 0.199 mm |
| Rabbit #33 | 0.191 mm | 0.210 mm |
| Rabbit #35 | 0.294 mm | 0.3012 mm |
| Rabbit #36 | 0.334 mm | 0.568 mm |

TABLE 19
Average size of small laser burn spots (<300 micron).
Large secondary were excluded

| | Experiment eye | Control eye |
|---|---|---|
| N = 6 | mean ± SD | mean ± SD |
| Rabbit #27 | 0.126 ± 0.048 | 0.178 ± 0.068 |
| Rabbit #28 | 0.134 ± 0.067 | 0.1245 ± 0.072 |
| Rabbit #32 | 0.154 ± 0.069 | 0.175 ± 0.043 |
| Rabbit #33 | 0.126 ± 0.059 | 0.133 ± 0.058 |
| Rabbit #35 | 0.153 ± 0.079 | 0.182 ± 0.053 |
| Rabbit #36 | 0.168 ± 0.056 | 0.206 ± 0.069 |

Table 19 shows that average size of small local laser burns were smaller in the right eye (mesenchymal cells) as compared to control left eye (saline alone). Differences between left and right eyes did not reach accepted significance level (P>0.05).

The authors were unable to find transplanted human cells on paraffin sections: neither by using Giemsa staining, nor by using anti-BrdU method. Both immunofluorescence and peroxidase microscopy failed to reveal human BrdU-positive cells. Auto fluorescence level and endogenous peroxidase activity were too high making it impossible to detect specific staining. Authors are currently trying to overcome these technical difficulties.

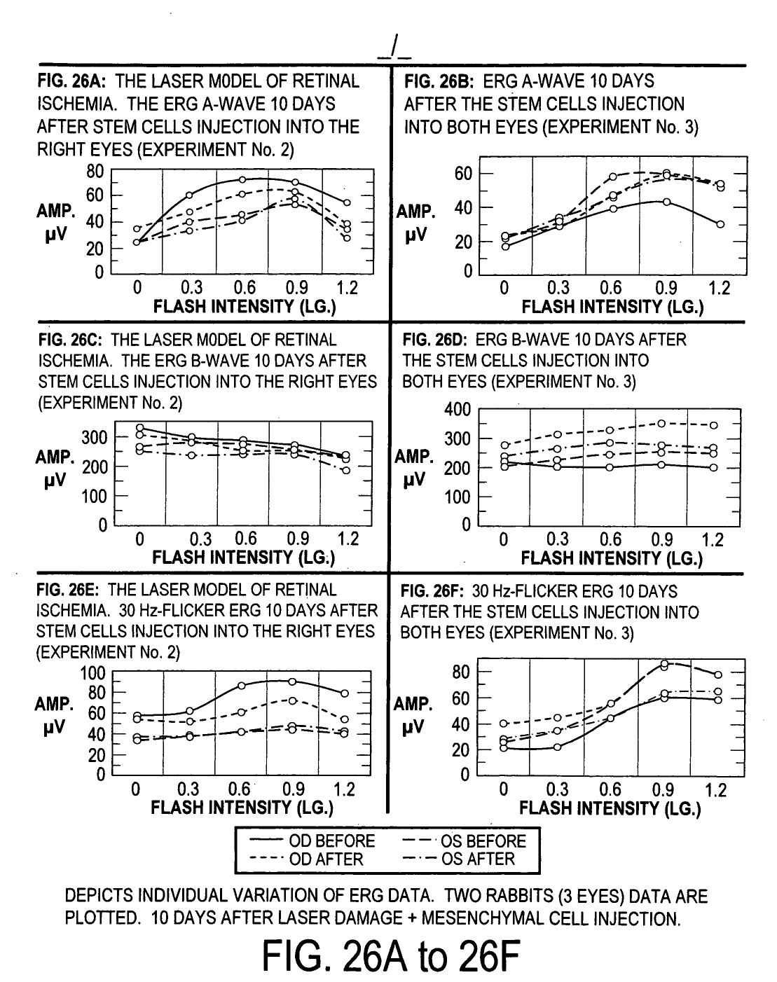

FIG. 30 depicts Rabbit #27. A,B,C,D,E—small isolated laser burn spots. F—large deep lesion caused by coagulated artery (interrupted blood flow).

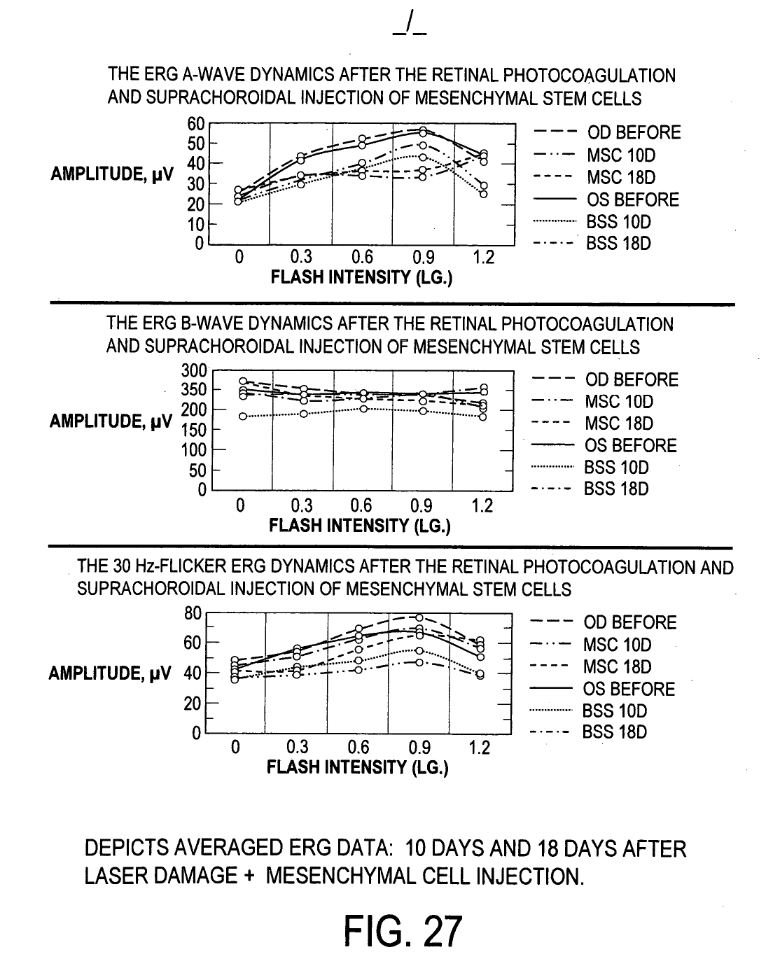

FIG. 31 shows Rabbit #28. Scale bar=200 micron. Typical small laser spot is shown. Arrows indicate spot that was included in analysis (the spot must not contain any photoreceptor cells).

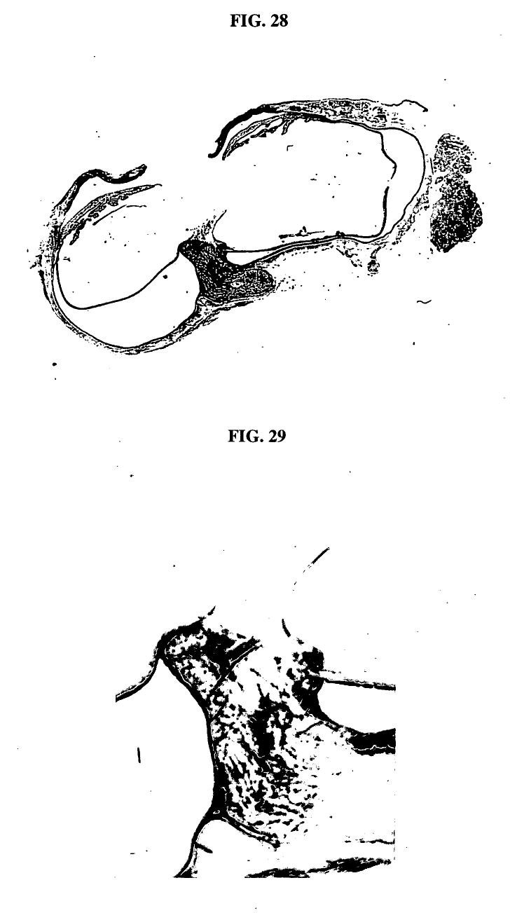

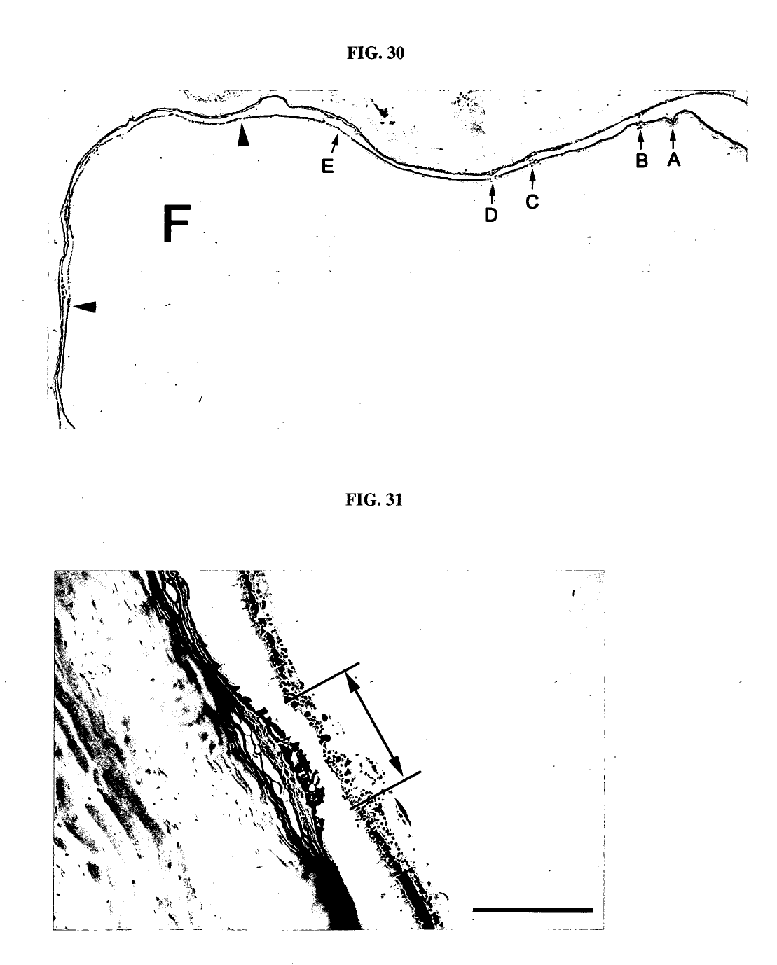

FIG. 32 shows results from Rabbit #27. Same as Microphoto 30, but at higher magnification. Large secondary lesion caused by occluded small artery. Photoreceptor cells are gone. Scale bar=200 micron.

FIG. 32 shows Rabbit #27. A site of mesenchymal cell injection is shown by red arrow. Blue-stained mass is presumably formed by transplanted human cells.

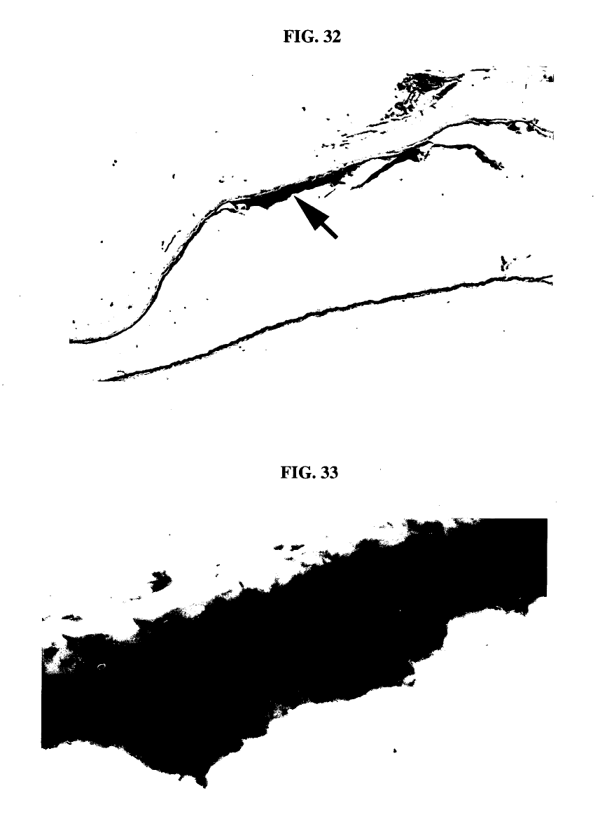

FIG. 33 depicts a magnified portion of FIG. 32.

1. A method for treating a side effect resulting from the application of laser therapy in the treatment of an eye disorder comprising:
a. treating an eye disorder in a patient with a laser, wherein said treating produces a side effect in the eye of said patient; and
b. administering a therapeutic amount of stem cells to said patient, wherein said administering treats said side effect.

2. The method of claim 1, wherein said eye disorder is selected from the group consisting diabetic retinopathy, macular edema, age-related macular degeneration, retinal tears, retinal holes, retinal detachment, retinal vein occlusion, dot and blot hemorrhages, microaneurysms, exudates, glaucoma, vitreous hemorrhage, inflammatory optic neuropathies, post-cataract complications, endophthalmitis, infectious diseases, ocular ischemia syndrome, peripheral retinal degenerations, toxic retinopathies, tumors, choroidal tumors, choroidal disorders, vitreous disorders and traumatic injury.

3. The method of claim 1, wherein said side effect is selected from the group consisting of vision impairment, destruction of retinal tissue and bleeding.

4. The method of claim 3, wherein said vision impairment is selected from the group consisting of blindness, lost or decreased peripheral vision, lost or decreased night vision, lost or decreased.

5. The method of claim 1, wherein said stem cells are selected from the group consisting of mesenchymal cells, neural stem cells, bulbus olfactorious cells, olfactory ensheathing cells and combinations thereof.

6. The method of claim 1, wherein said stem cells are administered according to a route selected from intravitreally, retrobulbarly, intravenously, suprachoroidally, and combinations thereof.

7. The method of claim 5, wherein said mesenchymal cells are derived from a source selected from bone marrow, adipose, peripheral blood, umbilical cord blood, umbilical cord, dermis, Wharton's jelly, hair follicle, periosteum, muscle tissue, uterine endometrium, amniotic fluid, tooth pulp, and combinations thereof.

8. The method of claim 1, wherein said laser therapy comprises photocoagulation.

9. The method of claim 8, wherein said photocoagulation is selected from the group consisting of focal photocoagulation, pan-retinal photocoagulation, and combinations thereof.

10. A method for treating a side effect resulting from photocoagulation therapy of an eye disorder, comprising:
a. administering photocoagulation therapy in the treatment of an eye disorder in a patient, wherein said photocoagulation therapy produces a side effect;
b. administering to said patient an amount of stem cells sufficient to treat said side effect.

11. The method of claim 10, wherein said eye disorder is selected from the group consisting of diabetic retinopathy, macular edema, age-related macular degeneration, retinal tears, retinal holes, retinal detachment, retinal vein occlusion, dot and blot hemorrhages, microaneurysms, exudates, glaucoma, vitreous hemorrhage, inflammatory optic neuropathies, post-cataract complications, endophthalmitis, infectious diseases, ocular ischemia syndrome, peripheral retinal degenerations, toxic retinopathies, tumors, choroidal tumors, choroidal disorders, vitreous disorders and traumatic injury.

12. The method of claim 10, wherein said stem cells are selected from the group consisting of mesenchymal cells, neural stem cells, bulbus olfactorious cells, olfactory ensheathing cells and combinations thereof.

13. The method of claim 10, wherein said stem cells are administered according to a route selected from intravitreally, retrobulbarly, intravenously, suprachoroidally, and combinations thereof.

14. The method of claim 12, wherein said mesenchymal cells are derived from a source selected from bone marrow, adipose, peripheral blood, umbilical cord blood, umbilical cord, dermis, Wharton's jelly, hair follicle, periosteum, muscle tissue, uterine endometrium, amniotic fluid, tooth pulp, and combinations thereof.

15. The method of claim 10, wherein said photocoagulation is selected from the group consisting of focal photocoagulation, pan-retinal photocoagulation, and combinations thereof.
